(19) United States
(12) Patent Application Publication
(10) Pub. No.: US 2004/0248791 A1
(43) Pub. Date: Dec. 9, 2004

(54) Insect g protein-coupled receptor genes and uses thereof

(76) Inventors: Eric Spana, Raleigh, NC (US); Kim Kamdar, Boston, MA (US); Lynn Stam, Raleigh, NC (US); Scott Valentine, Research Park Triangle, NC (US); Charles Michael Griswold, Cary, NC (US)

Correspondence Address: SYNGENTA BIOTECHNOLOGY, INC., PATENT DEPARTMENT, 3054 CORNWALLIS ROAD, P.O. BOX 12257, RESEARCH TRIANGLE PARK, NC, 27709-2257, US

(21) Appl. No.: 10/489,425

(22) PCT Filed: Dec. 18, 2002
(86) PCT No.: PCT/US02/40525

Related U.S. Application Data
(60) Provisional application No. 60/341,512, filed on Dec. 18, 2001.

(51) Int. Cl. C12Q 1/68; C07H 21/04; C07K 14/705; C12N 5/06; C12N 5/04

(52) U.S. Cl. 514/12; 530/350; 536/23.5; 435/6; 435/69.1; 435/320.1; 435/252.3; 435/348; 435/419

(57) ABSTRACT

The present invention provides isolated nucleic acids encoding insect G protein-coupled receptor (GPCR) polypeptides, isolated insect GPCR polypeptides, and uses thereof. Further provided are recombinant proteins and methods for identifying inhibitors to these proteins. The disclosed insect GPCR nucleic acids and polypeptides can be used in screening assays to identify modulating compounds. Protein inhibitors active in the methods disclosed herein are useful as insecticidal, ectoparasiticidal, antiparasitic, anthenenthic and acaracidal agents.

This application claims the benefit of U.S. Provisional Application No. 60/341,512 filed Dec. 18, 2001, which is hereby incorporated by reference in its entirety.

### FIELD OF THE INVENTION

The present invention generally relates to G protein-coupled receptor (GPCR) genes from Drosophila melanogaster, Heliothis virescens, or Manduca sexta. More particularly, the present invention provides novel GPCR nucleic acid and polypeptide sequences, chimeric genes comprising the disclosed GPCR sequences, antibodies that specifically recognize the disclosed GPCR polypeptides, modulators of GPCR nucleic acids and polypeptides, and uses thereof.

### Table of Abbreviations

ATCC American Tissue Culture Collection

dsRNA double-stranded RNA

dsRNAi double-stranded RNA interference

FCS Fluorescence Correlation Spectroscopy

GPCR G protein-coupled receptor

GST glutathione S transferase

HMM Hidden Markov Model

IPM integrated pest management

PCR polymerase chain reaction

PEG polyethylene glycol

RACE rapid amplification of cDNA ends

SELDI-TOF MS Surface-Enhanced Laser Desorption/Ionization Time-of-Flight Mass Spectroscopy

Sf9 cells Spodoptera frugiperda cells

SPR Surface Plasmon Resonance

### BACKGROUND

Insects contribute or cause many human and animal diseases, and are responsible for substantial agricultural and property damage. The societal costs associated with insect pests in dollars, time, and suffering are monumental. To combat these problems, insecticidal compounds have been developed and employed. The total worldwide market size for insecticide crop protection is over $5 billion, and insecticide products comprise approximately 32% of world consumption of pesticides.

Insecticide development has been guided predominantly by lead-finding efforts for new chemical structures. According to this strategy, chemical derivatization of a known insecticide is performed, and the synthesized compounds are analyzed for insecticidal activity. An alternative approach relies on methods for detecting molecular interactions between a candidate compound and a target molecule. An ideal target molecule is precisely regulated during insect development, such that modulation of the activity or level of activity of the target molecule results in organismal lethality. High throughput screening methods have enabled rapid screening of diverse and populous compound libraries for an ability to interact with a target molecule. The novel modulators discovered by such methods are useful as insecticides.

A target molecule can be further selected based on modulation of the target molecule activity that results in lethality during larval development. The insect life cycle requires successive larval or nymph stages that are devoted to growth such that the animal can increase mass by several thousand-fold. To sustain this growth, immature insects feed unabated for prolonged periods, and thus are particularly deleterious to agricultural crops during this developmental stage.

A crucial factor in the proper development and physiology of multi-cellular organisms is the correct interaction of a cell with its environment. One mechanism for an environmental signal to be recognized by a cell is by a plasma membrane localized receptor protein. Of the many categories of receptors found on the plasma membrane, one type, the G protein-coupled receptor (GPCR) has been shown to transduce a wide variety of extracellular signals into the cell cytoplasm via a reversible coupling to heterotrimeric G proteins. GPCRs proteins span the plasma membrane seven times in such a conformation as to have their amino terminus on the outside of the cell followed by seven consecutive transmembrane domains and their carboxy terminus on the cytoplasmic side of the membrane. Interacting molecules or ligands interact with the amino terminus and/or the three extracellular loops to produce a conformational change that allows the cytoplasmic portions of the receptor to alter its binding to the cytoplasmic G proteins. G protein-coupled receptors such as those in the present invention are characterized by the ability of a ligand to promote the formation of a high-affinity ternary complex between the ligand, the receptor, and an intracellular G protein. This complex is formed in the presence of physiological concentrations of GTP, and results in the dissociation of the alpha subunit of the G protein from the beta and gamma subunits of the G protein, which further results in a functional response, i.e., activation of downstream effectors such as adenylyl cyclase or phospholipase C.

The ability of a GPCR to produce an intracellular response to an extracellular stimulus has made this class of protein highly valued in drug and chemical discovery. It has been estimated that as much as 60% of all pharmaceutical drugs on the market are involved either directly or indirectly with GPCR signaling. Some examples of these drugs include Loratadine (Claritin®) an H1-histamine receptor antagonist and Theophylin (TheoDur®) an adenosine receptor antagonist. Complex physiological responses in humans such as modulation of pain perception and appetite regulation have been identified as consequences of GPCR signaling.

Like humans, many insect developmental and physiological processes are regulated by GPCR signaling. For example, the serotonin receptor 5-HT2 is required for embryonic development in Drosophila (Colas et al. (1999) Mech. Dev. 87: 67-76). Another GPCR called “flamingo” has multiple roles in development including dendrite formation and cell polarity (Gao et al. (2000) Neuron 28: 91-101; and Usui et at. (1999) Cell 98: 585-595). Also, fluid secretion in the insect renal gland, the malpighian tubule, has been shown to be directed by multiple GPCRs (Pietrantonio et al. (2001) Insect Mol Biol 10(4):357-69). These multiple functions throughout the life of an insect are why these GPCRs make excellent targets for insecticide discovery. Nature itself has shown that the toxin of the Black Widow spider, Latrotoxin, acts through a GPCR called latrophilin to initiate its effect.

There exists a continuing demand for insecticides that show improved efficacy and new modes of action. To this end, the present invention discloses a functional characterization of G protein-coupled receptors during Drosophila development.

### SUMMARY OF INVENTION

The present invention discloses isolated insect GPCR polypeptides and isolated nucleic acid molecules encoding the same. Preferably, an isolated insect GPCR polypeptide, or functional portion thereof, comprises a polypeptide encoded by the nucleic acid molecule of any one of odd numbered sequences of SEQ ID NOs:1-107; a polypeptide encoded by a nucleic acid molecule that is substantially identical to any one of odd numbered sequences of SEQ ID NOs:1-107; a polypeptide having an amino acid sequence of any one of even numbered sequences of SEQ ID NOs:2-108; a polypeptide that is a biological equivalent of any one of even numbered sequences of SEQ ID NOs:2-108; or a polypeptide that is immunologically cross-reactive with an antibody that shows specific binding with a polypeptide comprising some or all amino acids of any one of even numbered sequences of SEQ ID NOs:2-108.

In one embodiment, the present invention provides an isolated nucleic acid molecule comprising a nucleotide sequence, the complement of which hybridizes under stringent conditions to a sequence selected from the group consisting of the odd numbered SEQ ID NOs:1-107. In another embodiment, the present invention provides an isolated nucleic acid molecule comprising a nucleotide sequence that encodes a protein comprising an amino acid sequence having at least 60%, preferably 70%, more preferably 80%, still more preferably 90%, even more preferably 95%, and most preferably 99-100% sequence identity to an amino acid sequence selected from the group consisting of the even numbered SEQ ID NOs:2-108.

The present invention also provides a chimeric construct comprising promoter operatively linked to a nucleic acid molecule according to the present invention, wherein the promoter is preferably functional in a eukaryote, wherein the promoter is preferably heterologous to the nucleic acid molecule. More preferably, the promoter is functional in a plant.

The present invention further provides a recombinant vector comprising a chimeric construct according to the present invention, wherein said vector is capable of being stably transformed into a host cell. The present invention still further provides a host cell comprising a nucleic acid molecule according to the present invention, wherein said nucleic acid molecule is preferably expressible in the cell. The host cell is preferably selected from the group consisting of a plant cell, a yeast cell, an insect cell, and a prokaryotic cell. The present invention additionally provides a plant or seed comprising a plant cell according to the present invention.

The present invention also provides proteins essential for growth of an insect. In one embodiment, the present invention provides an isolated protein comprising an amino acid sequence having at least 60%, preferably 70%, more preferably 80%, still more preferably 90%, even more preferably 95%, and most preferably 99-100% sequence identity to an amino acid sequence selected from the group consisting of the even numbered SEQ ID NOs:2-108. In accordance with another embodiment, the present invention also relates to the recombinant production of proteins of the invention and methods of using the proteins of the invention in assays for identifying compounds that interact with the protein.

In another aspect of the invention, a method is provided for detecting a nucleic acid molecule that encodes an insect GPCR polypeptide. According to the method, a biological sample having nucleic acid material is hybridized under stringent hybridization conditions to an insect GPCR nucleic acid molecule of the present invention. Such hybridization enables a nucleic acid molecule of the biological sample and the insect GPCR nucleic acid molecule to form a detectable duplex structure. Preferably, the insect GPCR nucleic acid molecule includes some or all nucleotides of any one of odd numbered sequences of SEQ ID NOs:1-107. The present invention further teaches an antibody that specifically recognizes an insect GPCR polypeptide. Preferably, the antibody recognizes some or all amino acids of any one of even numbered sequences of SEQ ID NOs:2-108. A method for producing an insect GPCR antibody is also disclosed, and the method comprises recombinantly or synthetically producing an insect GPCR polypeptide, or portion thereof, as set forth in any one of even numbered sequences of SEQ ID NOs:2-108; formulating the insect GPCR polypeptide so that it is an effective immunogen; immunizing an animal with the formulated polypeptide to generate an immune response that includes production of insect GPCR antibodies; and collecting blood serum from the immunized animal containing antibodies that specifically recognize an insect GPCR polypeptide. Antibody-producing cells can be optionally fused with an immortal cell line whereby a monoclonal antibody that specifically recognizes an insect GPCR polypeptide can be selected.

A method is also provided for detecting a level of insect GPCR polypeptide using an antibody that recognizes an insect GPCR polypeptide of any of even numbered sequences of SEQ ID NOs:2-108. According to the method, a biological sample is obtained from an experimental subject and a control subject, and an insect GPCR polypeptide is detected in the sample by immunochemical reaction with the insect GPCR antibody. Preferably, the antibody recognizes amino acids of any one of even numbered sequences of SEQ ID NOs:2-108; and is prepared according to a method of the present invention for producing such an antibody.

The present invention further discloses a method for identifying a compound that modulates GPCR function. The method comprises: (a) exposing an isolated insect GPCR polypeptide of any one of even numbered sequences of SEQ ID NOs:2-108 to one or more compounds, and (b) assaying binding of a compound to the isolated insect GPCR polypeptide. A compound is selected that demonstrates specific binding to the isolated insect GPCR polypeptide. Preferably, the modulator is a chemical compound, a protein, a peptide, a nucleic acid, or an antibody, and was prepared according to a method disclosed herein.

The present invention also provides a method for identifying an insecticidal compound that modulates GPCR function. The method comprises: (a) isolating an insect GPCR polypeptide of any one of even numbered SEQ ID NOs:2-108, wherein modulation of the insect GPCR polypeptide confers lethality of an insect; (b) exposing the isolated insect GPCR polypeptide to a plurality of substances; (c) assaying binding of a substance to the isolated GPCR polypeptide; and (d) selecting a substance that demonstrates specific binding to the isolated insect GPCR polypeptide. Preferably, the modulator is a chemical compound, a protein, a peptide, a nucleic acid, or an antibody, and was prepared according to a method disclosed herein.

The present invention provides nucleic acid molecules isolated from insects comprising nucleotide sequences that encode proteins essential for insect viability, preferably G protein-coupled receptors. This knowledge is exploited to provide novel insecticidal modes of action. The critical role in insect growth of the proteins encoded by each of the nucleotide sequences of the invention implies that chemicals that inhibit the function of any one of these proteins in insects are likely to have detrimental effects on insects and are potentially good insecticide candidates. Thus, the proteins encoded by the essential nucleotide sequences provide the bases for assays designed to easily and rapidly identify novel insecticides.

The present invention therefore provides methods of using a purified protein encoded by any one of the nucleotide sequences described below to identify inhibitors thereof, which can then be used as insecticides to suppress the growth of undesirable insects, e.g. in fields where crops are grown, particularly agronomically important crops such as maize and other cereal crops such as wheat, oats, rye, sorghum, rice, barley, millet, turf and forage grasses, and the like, as well as cotton, sugar cane, sugar beet, oilseed rape, and soybeans.

According to another aspect, the present invention provides a method of identifying an insecticidal compound, comprising: (a) combining a polypeptide comprising an amino acid sequence at least 90% identical to an amino acid sequence selected from the group consisting of the even numbered SEQ ID NOs:2-108 with a compound to be tested for the ability to bind to said polypeptide, under conditions conducive to binding; (b) selecting a compound identified in (a) that binds to said polypeptide; (c) applying a compound selected in (b) to a plant to test for insecticidal activity; and (d) selecting a compound identified in (c) that has insecticidal activity. Preferably, the polypeptide comprises an amino acid sequence at least 95% identical to an amino acid sequence selected from the group consisting of the even numbered SEQ ID NOs:2-108. More preferably, the polypeptide comprises an amino acid sequence at least 99% identical to an amino acid sequence selected from the group consisting of the even numbered SEQ ID NOs:2-108. Most preferably, the polypeptide comprises an amino acid sequence selected from the group consisting of the even numbered SEQ ID NOs:2-108. The present invention also provides a method for killing or inhibiting the growth or viability of an insect, comprising applying to the insect, or applying to plant tissue, an insecticidal compound identified according to this method.

According to yet another aspect, the present invention provides a method of identifying an insecticidal compound, comprising: (a) combining a polypeptide comprising an amino acid sequence at least 90% identical to an amino acid sequence selected from the group consisting of the even numbered SEQ ID NOs:2-108 with a compound to be tested for the ability to inhibit the activity of said polypeptide, under conditions conducive to inhibition; (b) selecting a compound identified in (a) that inhibits the activity of said polypeptide; (c) applying a compound selected in (b) to an insect to test for insecticidal activity; and (d) selecting a compound identified in (c) that has insecticidal activity. Preferably, the polypeptide comprises an amino acid sequence at least 95% identical to an amino acid sequence selected from the group consisting of the even numbered SEQ ID NOs:2-108. More preferably, the polypeptide comprises an amino acid sequence at least 99% identical to an amino acid sequence selected from the group consisting of the even numbered SEQ ID NOs:2-108. Most preferably, the polypeptide comprises an amino acid sequence selected from the group consisting of the even numbered SEQ ID NOs:2-108. The present invention also provides a method for killing or inhibiting the growth or viability of an insect, comprising applying to the insect, or to plant tissue, the insecticidal compound identified according to this method.

The present invention still further provides a method for killing or inhibiting the growth or viability of an insect, comprising inhibiting expression in said insect of a protein having at least 60%, preferably 70%, more preferably 80%, still more preferably 90%, even more preferably 95%, and most preferably 99-100% sequence identity to an amino acid sequence selected from the group consisting of the even numbered SEQ ID NOs:2-108.

The present invention further provides a method for preventing or treating an insect infestation of a plant, the method comprising: (a) preparing an insecticidal composition that is a modulator of an insect GPCR set forth as any one of even-numbered SEQ ID NOs:2-108; and (b) contacting an effective dose of the insecticidal composition with a plant, whereby an insect infestation of the plant is prevented or abrogated. Preferably, the insecticidal composition comprises a chemical compound, a protein, a peptide, a nucleic acid, or an antibody, and was prepared according to a method disclosed herein. Preferably, the insect infestation is abrogated by lethality of the insect. In one embodiment, the insecticidal composition also displays nematicide activity, such that contacting an effective dose of the insecticidal composition with a plant prevents or abrogates a nematode infestation of the plant.

The present invention further provides a method for preventing or abrogating an insect infestation of a plant, the method comprising: (a) expressing in a plant an insect GPCR modulator that modulates the activity of an insect GPCR polypeptide of any one of even-numbered SEQ ID NOs:2-108, whereby an insect infestation of a plant is prevented or abrogated. Preferably, the insecticidal composition comprises a protein, a peptide, a nucleic acid, or an antibody. In one embodiment, the insecticidal composition additionally displays nematicidal activity, such that expression of insect GPCR modulator in a plant prevents or abrogates a nematode infestation of the plant. The present invention further embodies plants, plant tissues, plant seeds, and plant cells that express an insect GPCR modulator and that are therefore able to inhibit plant parasitic nematode infestation.

Accordingly, it is an object of the present invention to provide novel insect GPCR nucleic acids and polypeptides, and novel methods relating thereto. This object is achieved in whole or in part by the present invention.

An object of the invention having been stated above, other objects and advantages of the present invention will become apparent to those skilled in the art after a study of the following description of the invention, and non-limiting Examples.

### Brief Description of Sequences in the Sequence Listing

Odd-numbered SEQ ID NOs:1-107 are nucleotide sequences encoding GPCRs described in Table 1.

Even-numbered SEQ ID NOs:2-108 are protein sequences encoded by the immediately preceding nucleotide sequence, e.g., SEQ ID NO:2 is the protein encoded by the nucleotide sequence of SEQ ID NO:1, SEQ ID NO:4 is the protein encoded by the nucleotide sequence of SEQ ID NO:3, etc.

SEQ ID NO:109 is an octopamine forward degenerate primer.

SEQ ID NO:110 is an octopamine reverse degenerate primer.

SEQ ID NO:111 is a 5′ RACE primer.

SEQ ID NO:112 is a 3′ RACE primer.

TABLE 1
Sequence Listing Summary

| SEQ ID NOS. | Inventor's Reference |
|---|---|
| 1-2 | Adenosine_99E1 |
| 3-4 | Allatostatin_3E1 |
| 5-6 | Gastrin_2C3 |
| 7-8 | wormlike_4F9 |
| 9-10 | Dopamine_6D7 |
| 11-12 | Lymnokinin_11D9 |
| 13-14 | mthlike1_15A |
| 15-16 | gastrin_17E3 |
| 17-18 | CCK-X_26A1 |
| 19-20 | neuroYY_26B1 |
| 21-22 | GRHR_27A2 |
| 23-24 | Bombesin_42A10 |
| 25-26 | Latrophillin_44D4 |
| 27-28 | wormlike_47E |
| 29-30 | Calcitonin_49F9 |
| 31-32 | DiureticHor2_51A1 |
| 33-34 | mthlike3_54B16 |
| 35-36 | bombesin_54D4 |
| 37-38 | 5-HT1A_56A |
| 39-40 | mAcR-60C |
| 41-42 | Adrenergic_60D1 |
| 43-44 | mthlike9_61C |
| 45-46 | mthlike10_61C1 |
| 47-48 | wormlike_62 |
| 49-50 | wormlike_63A3 |
| 51-52 | adrenergic_64C |
| 53-54 | lymnokinin_64D3 |
| 55-56 | mthlike2_64D4 |
| 57-58 | GRHR_69B |
| 59-60 | somatostatin_75C1 |
| 61-62 | NeuroYlike_77A1 |
| 63-64 | Secretagogue_79A |
| 65-66 | 5-HT2_82C4 |
| 67-68 | NeuroYYlike_83 |
| 69-70 | mAcChR_84E8 |
| 71-72 | Takr-86C_86C3 |
| 73-74 | mthlike5_87A |
| 75-76 | GrowthHormone_87F1 |
| 77-78 | neurotensin_88B1 |
| 79-80 | DopR_88B1 |
| 81-82 | Octopamine_90C2 |
| 83-84 | FSHlike_96E5 |
| 85-86 | Histamine_97B |
| 87-88 | NeuropepYR_97E1 |
| 89-90 | Galanin_98E2 |
| 91-92 | Takr-99D_99D1 |
| 93-94 | 5-HT7_100A2 |
| 95-96 | He6Receptor_100B1 |
| 97-98 | Fsh 90c2 |
| 99-100 | He6Receptor2 |
| 101-102 | Prostaglandin 74F1 |
| 103-104 | Oct-TyrR 79D1 |
| 105-106 | Octopamine type 2 Heliothis |
| 107-108 | Diuretic Hormone Manduca |
| 109 | Octopamine forward degenerate |
| | primer |
| 110 | Octopamine reverse degererate |
| | primer |
| 111 | 5′ RACE Primer |
| 112 | 3′ RACE primer |

### DETAILED DESCRIPTION OF THE INVENTION

I. Definitions

While the following terms are believed to be well understood by one of ordinary skill in the art, the following definitions are set forth to facilitate explanation of the invention.

The term “nucleic acid molecule” refers to deoxyribonucleotides or ribonucleotides and polymers thereof in either single- or double-stranded form. Unless specifically limited, the term encompasses nucleic acids containing known analogues of natural nucleotides which have similar properties as the reference natural nucleic acid. Unless otherwise indicated, a particular nucleotide sequence also implicitly encompasses conservatively modified variants thereof (e.g., degenerate codon substitutions), complementary sequences, subsequences, elongated sequences, as well as the sequence explicitly indicated. The terms “nucleic acid molecule” or “nucleotide sequence” can also be used in place of “gene”, “cDNA”, or “mRNA”. Nucleic acids can be derived from any source, including any organism.

The term “isolated”, as used in the context of a nucleic acid molecule, indicates that the nucleic acid molecule exists apart from its native environment and is not a product of nature. An isolated DNA molecule can exist in a purified form or can exist in a non-native environment such as a transgenic host cell.

The term “purified”, when applied to a nucleic acid, denotes that the nucleic acid is essentially free of other cellular components with which it is associated in the natural state. Preferably, a purified nucleic acid molecule is a homogeneous dry or aqueous solution. The term “purified” denotes that a nucleic acid gives rise to essentially one band in an electrophoretic gel. Particularly, it means that the nucleic acid is at least about 50% pure, more preferably at least about 85% pure, and most preferably at least about 99% pure.

The term “substantially identical”, in the context of two nucleotide sequences, refers to two or more sequences or subsequences that have at least 60%, preferably about 70%, more preferably about 80%, more preferably about 90-95%, and most preferably about 99% nucleotide identity, when compared and aligned for maximum correspondence, as measured using one of the following sequence comparison algorithms (described herein below under the heading “Nucleotide and Amino Acid Sequence Comparisons” or by visual inspection. Preferably, the substantial identity exists in nucleotide sequences of at least 50 residues, more preferably in nucleotide sequence of at least about 100 residues, more preferably in nucleotide sequences of at least about 150 residues, and most preferably in nucleotide sequences comprising complete coding sequences. In one aspect, polymorphic sequences can be substantially identical sequences. The term “polymorphic” refers to the occurrence of two or more genetically determined alternative sequences or alleles in a population. An allelic difference can be as small as one base pair.

Another indication that two nucleotide sequences are substantially identical is that the two molecules specifically or substantially hybridize to each other under stringent conditions. In the context of nucleic acid hybridization, two nucleic acid sequences being compared can be designated a “probe” and a “target”. A “probe” is a reference nucleic acid molecule, and a “‘target” is a test nucleic acid molecule, often found within a heterogeneous population of nucleic acid molecules. A “target sequence” is synonymous with a “test sequence”.

A preferred nucleotide sequence employed for hybridization studies or assays includes probe sequences that are complementary to or mimic at least an about 14 to 40 nucleotide sequence of a nucleic acid molecule of the present invention. Preferably, probes comprise 14 to 20 nucleotides, or even longer where desired, such as 30, 40, 50, 60, 100, 200, 300, or 500 nucleotides or up to the full length of any of those set forth as odd numbered sequences SEQ ID NOs:1-107. Such fragments can be readily prepared by, for example, directly synthesizing the fragment by chemical synthesis, by application of nucleic acid amplification technology, or by introducing selected sequences into recombinant vectors for recombinant production.

The phrase “hybridizing specifically to” refers to the binding, duplexing, or hybridizing of a molecule only to a particular nucleotide sequence under stringent conditions when that sequence is present in a complex nucleic acid mixture (e.g., total cellular DNA or RNA).

The phrase “hybridizing substantially to” refers to complementary hybridization between a probe nucleic acid molecule and a target nucleic acid molecule and embraces minor mismatches that can be accommodated by reducing the stringency of the hybridization media to achieve the desired hybridization.

“Stringent hybridization conditions” and “stringent hybridization wash conditions” in the context of nucleic acid hybridization experiments such as Southern and Northern blot analysis are both sequence- and environment-dependent. Longer sequences hybridize specifically at higher temperatures. An extensive guide to the hybridization of nucleic acids is found in Tijssen (1993) Laboratory Techniques in Biochemistry and Molecular Biology—Hybridization with Nucleic Acid Probes, part I chapter 2, Elsevier, New York, N.Y. Generally, highly stringent hybridization and wash conditions are selected to be about 5° C. lower than the thermal melting point (Tₘ) for the specific sequence at a defined ionic strength and pH. Typically, under “stringent conditions” a probe will hybridize specifically to its target subsequence, but to no other sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for Southern or Northern Blot analysis of complementary nucleic acids having more than about 100 complementary residues is overnight hybridization in 50% formamide with 1 mg of heparin at 42° C. An example of highly stringent wash conditions is 15 minutes in 0.1×SSC, SM NaCl at 65° C. An example of stringent wash conditions is 15 minutes in 0.2×SSC buffer at 65° C. (See Sambrook et al., eds (1989) Molecular Cloning: A Laboratory Manual, Cold Spring Harbor Laboratory Press, Cold Spring Harbor, N.Y. for a description of SSC buffer). Often, a high stringency wash is preceded by a low stringency wash to remove background probe signal. An example of medium stringency wash conditions for a duplex of more than about 100 nucleotides, is 15 minutes in 1×SSC at 45° C. An example of low stringency wash for a duplex of more than about 100 nucleotides, is 15 minutes in 4-6×SSC at 40° C. For short probes (e.g., about 10 to 50 nucleotides), stringent conditions typically involve salt concentrations of less than about 1M Na⁺ ion, typically about 0.01 to 1M Na⁺ ion concentration (or other salts) at pH 7.0-8.3, and the temperature is typically at least about 30° C. Stringent conditions can also be achieved with the addition of destabilizing agents such as formamide. In general, a signal to noise ratio of 2-fold (or higher) than that observed for an unrelated probe in the particular hybridization assay indicates detection of a specific hybridization.

The following are examples of hybridization and wash conditions that can be used to clone homologous nucleotide sequences that are substantially identical to reference nucleotide sequences of the present invention: a probe nucleotide sequence preferably hybridizes to a target nucleotide sequence in 7% sodium dodecyl sulfate (SDS), 0.5M NaPO₄, 1 mM EDTA at 50° C. followed by washing in 2×SSC, 0.1% SDS at 50°c; more preferably, a probe and target sequence hybridize in 7% sodium dodecyl sulfate (SDS), 0.5M NaPO₄, 1 mM EDTA at 50° C. followed by washing in 1×SSC, 0.1% SDS at 50° C.; more preferably, a probe and target sequence hybridize in 7% sodium dodecyl sulfate (SDS), 0.5M NaPO₄, 1 mM EDTA at 50° C. followed by washing in 0.5×SSC, 0.1% SDS at 50° C.; more preferably, a probe and target sequence hybridize in 7% sodium dodecyl sulfate (SDS), 0.5M NaPO₄, 1 mM EDTA at 50° C. followed by washing in 0.1×SSC, 0.1% SDS at 50° C.; more preferably, a probe and target sequence hybridize in 7% sodium dodecyl sulfate (SDS), 0.5M NaPO₄, 1 mM EDTA at 50° C. followed by washing in 0.1×SSC, 0.1% SDS at 65° C.

A further indication that two nucleic acid sequences are substantially identical is that proteins encoded by the nucleic acids are substantially identical, share an overall three-dimensional structure, are biologically functional equivalents, or are immunologically cross-reactive. These terms are defined further under the heading “Polypeptides” herein below. Nucleic acid molecules that do not hybridize to each other under stringent conditions are still substantially identical if the corresponding proteins are substantially identical. This can occur, for example, when two nucleotide sequences are significantly degenerate as permitted by the genetic code.

The term “conservatively substituted variants” refers to nucleic acid sequences having degenerate codon substitutions wherein the third position of one or more selected (or all) codons is substituted with mixed-base and/or deoxyinosine residues (Batzer et al. (1991) Nuc Acids Res 19:5081; Ohtsuka et al. (1985) J Biol Chem 260:2605-2608; Rossolini et al. (1994) Mol Cell Probes 8:91-98).

The term “subsequence” refers to a sequence of nucleic acids that comprises a part of a longer nucleic acid sequence. An exemplary subsequence is a probe, described herein above, or a primer. The term “primer” as used herein refers to a contiguous sequence comprising about 8 or more deoxyribonucleotides or ribonucleotides, preferably 10-20 nucleotides, and more preferably 20-30 nucleotides of a selected nucleic acid molecule. The primers of the invention encompass oligonucleotides of sufficient length and appropriate sequence so as to provide initiation of polymerization on a nucleic acid molecule of the present invention.

The term “elongated sequence” refers to an addition of nucleotides (or other analogous molecules) incorporated into the nucleic acid. For example, a polymerase (e.g., a DNA polymerase) can add sequences at the 3′ terminus of the nucleic acid molecule. In addition, the nucleotide sequence can be combined with other DNA sequences, such as promoters, promoter regions, enhancers, polyadenylation signals, intronic sequences, additional restriction enzyme sites, multiple cloning sites, and other coding segments.

The term “complementary sequences”, as used herein, indicates two nucleotide sequences that comprise antiparallel nucleotide sequences capable of pairing with one another upon formation of hydrogen bonds between base pairs. As used herein, the term “complementary sequences” means nucleotide sequences which are substantially complementary, as can be assessed by the same nucleotide comparison set forth above, or is defined as being capable of hybridizing to the nucleic acid segment in question under relatively stringent conditions such as those described herein. A particular example of a complementary nucleic acid segment is an antisense oligonucleotide.

The term “gene” refers broadly to any segment of DNA associated with a biological function. A gene encompasses sequences including but not limited to a coding sequence, a promoter region, a cis-regulatory sequence, a non-expressed DNA segment that is a specific recognition sequence for regulatory proteins, a non-expressed DNA segment that contributes to gene expression, a DNA segment designed to have desired parameters, or combinations thereof. A gene can be obtained by a variety of methods, including cloning from a biological sample, synthesis based on known or predicted sequence information, and recombinant derivation of an existing sequence.

The term “gene expression” generally refers to the cellular processes by which a biologically active polypeptide is produced from a DNA sequence.

The present invention also encompasses chimeric genes comprising the disclosed GPCR sequences. The term “chimeric gene”, as used herein, refers to a promoter region operatively linked to a GPCR coding sequence, a nucleotide sequence producing an antisense RNA molecule, a RNA molecule having tertiary structure, such as a hairpin structure, or a double-stranded RNA molecule.

The term “operatively linked”, as used herein, refers to a promoter region that is connected to a nucleotide sequence in such a way that the transcription of that nucleotide sequence is controlled and regulated by that promoter region. Techniques for operatively linking a promoter region to a nucleotide sequence are known in the art.

The terms “heterologous gene”, “heterologous DNA sequence”, “heterologous nucleotide sequence”, “exogenous nucleic acid molecule”, or “exogenous DNA segment”, as used herein, each refer to a sequence that originates from a source foreign to an intended host cell or, if from the same source, is modified from its original form. Thus, a heterologous gene in a host cell includes a gene that is endogenous to the particular host cell but has been modified, for example by mutagenesis or by isolation from native cis-regulatory sequences. The terms also include non-naturally occurring multiple copies of a naturally occurring nucleotide sequence. Thus, the terms refer to a DNA segment that is foreign or heterologous to the cell, or homologous to the cell but in a position within the host cell nucleic acid wherein the element is not ordinarily found.

The term “transcription factor” generally refers to a protein that modulates gene expression by interaction with the cis-regulatory element and cellular components for transcription, including RNA Polymerase, Transcription Associated Factors (TAFs), chromatin-remodeling proteins, and any other relevant protein that impacts gene transcription.

The present invention further includes vectors comprising the disclosed nuclear sequences, including plasmids, cosmids, and viral vectors. The term “vector”, as used herein refers to a DNA molecule having sequences that enable its replication in a compatible host cell. A vector also includes nucleotide sequences to permit ligation of nucleotide sequences within the vector, wherein such nucleotide sequences are also replicated in a compatible host cell. A vector can also mediate recombinant production of a GPCR polypeptide, as described further herein below. A preferred host cell is a bacterial cell, an insect cell, a plant cell or mammalian cell.

Nucleic acids of the present invention can be cloned, synthesized, recombinantly altered, mutagenized, or combinations thereof. Standard recombinant DNA and molecular cloning techniques used to isolate nucleic acids are known in the art. Exemplary, non-limiting methods are described by Sambrook et al., eds (1989) Molecular Cloning, Cold Spring Harbor Laboratory Press, Cold Spring Harbor, N.Y.; by Silhavy et al. (1984) Experiments with Gene Fusions, Cold Spring Harbor Laboratory Press, Cold Spring Harbor, N.Y.; Sambrook, (2001) Molecular Cloning: a Laboratory Manual (3^{rd} ed), Cold Spring Harbor Laboratory Press, Cold Spring Harbor, N.Y.; by Ausubel et al. (1992) Current Protocols in Molecular Biology, John Wylie and Sons, Inc., New York, N.Y.; and by Glover, ed (1985) DNA Cloning: A Practical Approach, MRL Press, Ltd., Oxford, United Kingdom. Site-specific mutagenesis to create base pair changes, deletions, or small insertions are also known in the art as exemplified by publications. See, e.g., Adelman et al. (1983) DNA 2:183; Sambrook et al., eds (1989) Molecular Cloning, Cold Spring Harbor Laboratory Press, Cold Spring Harbor, N.Y.

Sequences detected by methods of the invention can be detected, subcloned, sequenced, and further evaluated by any measure known in the art using any method usually applied to the detection of a specific DNA sequence including but not limited to dideoxy sequencing, PCR, oligomer restriction (Saiki et al. (1985) Bio/Technology 3:1008-1012), allele-specific oligonucleotide (ASO) probe analysis (Conner et al. (1983) Proc Natl Acad Sci USA 80:278), and oligonucleotide ligation assays (OLAs) (Landgren et al. (1988) Science 241:1007). See also Landgren et al. (1988) Science 242:229-237.

The polypeptides provided by the present invention include the isolated polypeptides set forth as even sequences of SEQ ID NOs:2-108; polypeptides substantially identical to even numbered sequences of SEQ ID NOs:2-108; GPCR polypeptide fragments (preferably biologically functional fragments, e.g. the domains described herein), fusion proteins comprising the disclosed GPCR amino acid sequences, biologically functional analogs, and polypeptides that cross-react with an antibody that specifically recognizes a disclosed GPCR polypeptide.

The term “isolated”, as used in the context of a polypeptide, indicates that the polypeptide exists apart from its native environment and is not a product of nature. An isolated polypeptide can exist in a purified form or can exist in a non-native environment such as, for example, in a transgenic host cell.

The term “purified”, when applied to a polypeptide, denotes that the polypeptide is essentially free of other cellular components with which it is associated in the natural state. Preferably, a polypeptide is a homogeneous solid or aqueous solution. Purity and homogeneity are typically determined using analytical chemistry techniques such as polyacrylamide gel electrophoresis or high performance liquid chromatography. A polypeptide which is the predominant species present in a preparation is substantially purified. The term “purified” denotes that a polypeptide gives rise to essentially one band in an electrophoretic gel. Particularly, it means that the polypeptide is at least about 50% pure, more preferably at least about 85% pure, and most preferably at least about 99% pure.

The term “substantially identical” in the context of two or more polypeptide sequences is measured as polypeptide sequences having about 35%, or 45%, or preferably from 45-55%, or more preferably 55-65% of identical or functionally equivalent amino acids. Even more preferably, two or more “substantially identical” polypeptide sequences will have about 70%, or even more preferably about 80%, still more preferably about 90%, still more preferably about 95%, and most preferably about 99% identical or functionally equivalent amino acids. Percent “identity” and methods for determining identity are defined herein below under the heading “Nucleotide and Amino Acid Sequence Comparisons”.

Substantially identical polypeptides also encompass two or more polypeptides sharing a conserved three-dimensional structure. Computational methods can be used to compare structural representations, and structural models can be generated and easily tuned to identify similarities around important active sites or ligand binding sites. See Henikoff et al. (2000) Electrophoresis 21(9):1700-1706; Huang et al. (2000) Pac Symp Biocomput 230-241; Saqi et al. (1999) Bioinformatics 15(6):521-522; and Barton (1998) Acta Crystallogr D Biol Crystallogr 54:1139-1146.

The term “functionally equivalent” in the context of amino acid sequences is known in the art and is based on the relative similarity of the amino acid side-chain substituents. See Henikoff & Henikoff (2000) Adv Protein Chem 54:73-97. Relevant factors for consideration include side-chain hydrophobicity, hydrophilicity, charge, and size. For example, arginine, lysine, and histidine are all positively charged residues; that alanine, glycine, and serine are all of similar size; and that phenylalanine, tryptophan, and tyrosine all have a generally similar shape. By this analysis, described further herein below, arginine, lysine, and histidine; alanine, glycine, and serine; and phenylalanine, tryptophan, and tyrosine; are defined herein as biologically functional equivalents.

In making biologically functional equivalent amino acid substitutions, the hydropathic index of amino acids can be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte et al. (1982) J Mol Biol 157:105). It is known that certain amino acids can be substituted for other amino acids having a similar hydropathic index or score and still retain a similar biological activity. In making changes based upon the hydropathic index, the substitution of amino acids whose hydropathic indices are within ±2 of the original value is preferred, those which are within ±1 of the original value are particularly preferred, and those within ±0.5 of the original value are even more particularly preferred.

It is also understood in the art that the substitution of like amino acids can be made effectively on the basis of hydrophilicity. U.S. Pat. No. 4,554,101 states that the greatest local average hydrophilicity of a protein, as governed by the hydrophilicity of its adjacent amino acids, correlates with its immunogenicity and antigenicity, e.g., with a biological property of the protein. It is understood that an amino acid can be substituted for another having a similar hydrophilicity value and still obtain a biologically equivalent protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within ±2 of the original value is preferred, those which are within ±1 of the original value are particularly preferred, and those within ±0.5 of the original value are even more particularly preferred.

The present invention also encompasses GPCR polypeptide fragments or functional portions of a GPCR polypeptide. Such functional portion need not comprise all or substantially all of the amino acid sequence of a native GPCR gene product. The term “functional” includes any biological activity or feature of GPCR, including immunogenicity.

The present invention also includes longer sequences of a GPCR polypeptide, or portion thereof. For example, one or more amino acids can be added to the N-terminus or C-terminus of a GPCR polypeptide. Fusion proteins comprising GPCR polypeptide sequences are also provided within the scope of the present invention. Methods of preparing such proteins are known in the art.

The present invention also encompasses functional analogs of a GPCR polypeptide. Functional analogs share at least one biological function with a GPCR polypeptide. An exemplary function is immunogenicity. In the context of amino acid sequence, biologically functional analogs, as used herein, are peptides in which certain, but not most or all, of the amino acids can be substituted. Functional analogs can be created at the level of the corresponding nucleic acid molecule, altering such sequence to encode desired amino acid changes. In one embodiment, changes can be introduced to improve a biological function of the polypeptide, e.g., to improve the antigenicity of the polypeptide. In another embodiment, a GPCR polypeptide sequence is varied so as to assess the activity of a mutant GPCR polypeptide.

The present invention also encompasses recombinant production of the disclosed GPCR polypeptides. Briefly, a nucleic acid sequence encoding a GPCR polypeptide, or portion thereof, is cloned into an expression cassette, the cassette is introduced into a host organism, where it is recombinantly produced.

The term “expression cassette” as used herein means a DNA sequence capable of directing expression of a particular nucleotide sequence in an appropriate host cell, comprising a promoter operatively linked to the nucleotide sequence of interest which is operatively linked to termination signals. It also typically comprises sequences required for proper translation of the nucleotide sequence. The expression cassette comprising the nucleotide sequence of interest can be chimeric. The expression cassette can also be one which is naturally occurring but has been obtained in a recombinant form useful for heterologous expression.

The expression of the nucleotide sequence in the expression cassette can be under the control of a constitutive promoter or an inducible promoter which initiates transcription only when the host cell is exposed to some particular external stimulus. Exemplary promoters include Simian virus 40 early promoter, a long terminal repeat promoter from retrovirus, an action promoter, a heat shock promoter, and a metallothien protein. In the case of a multicellular organism, the promoter and promoter region can direct expression to a particular tissue or organ or stage of development. Suitable expression vectors which can be used include, but are not limited to, the following vectors or their derivatives: viruses such as vaccinia virus or adenovirus, baculovirus vectors, yeast vectors, bacteriophage vectors (e.g., lambda phage), plasmid and cosmid DNA vectors, and transposon-mediated transformation vectors.

The term “host cell”, as used herein, refers to a cell into which a heterologous nucleic acid molecule has been introduced. Transformed cells, tissues, or organisms are understood to encompass not only the end product of, a transformation process, but also transgenic progeny thereof.

A host cell strain can be chosen which modulates the expression of the inserted sequences, or modifies and processes the gene product in the specific fashion desired. For example, different host cells have characteristic and specific mechanisms for the translational and post-transactional processing and modification (e.g., glycosylation, phosphorylation of proteins). Appropriate cell lines or host systems can be chosen to ensure the desired modification and processing of the foreign protein expressed. Expression in a bacterial system can be used to produce a non-glycosylated core protein product. Expression in yeast will produce a glycosylated product. Expression in insect cells can be used to ensure “native” glycosylation of a heterologous protein.

Expression constructs are transfected into a host cell by any standard method, including electroporation, calcium phosphate precipitation, DEAE-Dextran transfection, liposome-mediated transfection, transposon-mediated transformation and infection using a retrovirus. The GPCR-encoding nucleotide sequence carried in the expression construct can be stably integrated into the genome of the host or it can be present as an extrachromosomal molecule.

Isolated polypeptides and recombinantly produced polypeptides can be purified and characterized using a variety of standard techniques that are known to the skilled artisan. See, e.g., Ausubel et al. (1992) Current Protocols in Molecular Biology, John Wylie & Sons, Inc., New York, N.Y.; Bodanszky, et al. (1976) Peptide Synthesis, John Wiley and Sons, Second Edition, New York, N.Y.; and Zimmer et al. (1993) Peptides, pp. 393-394, ESCOM Science Publishers, B. V.

I.C. Nucleotide and Amino Acid Sequence Comparisons The terms “identical” or percent “identity” in the context of two or more nucleotide or polypeptide sequences, refer to two or more sequences or subsequences that are the same or have a specified percentage of amino acid residues or nucleotides that are the same, when compared and aligned for maximum correspondence, as measured using one of the sequence comparison algorithms disclosed herein or by visual inspection.

The term “substantially identical” in regards to a nucleotide or polypeptide sequence means that a particular sequence varies from the sequence of a naturally occurring sequence by one or more deletions, substitutions, or additions, the net effect of which is to retain at least some of biological activity of the natural gene, gene product, or sequence. Such sequences include “mutant” sequences, or sequences wherein the biological activity is altered to some degree but retains at least some of the original biological activity. The term “naturally occurring”, as used herein, is used to describe a composition that can be found in nature as distinct from being artificially produced by man. For example, a protein or nucleotide sequence present in an organism, which can be isolated from a source in nature and which has not been intentionally modified by man in the laboratory, is naturally occurring.

For sequence comparison, typically one sequence acts as a reference sequence to which test sequences are compared. When using a sequence comparison algorithm, test and reference sequences are entered into a computer program, subsequence coordinates are designated if necessary, and sequence algorithm program parameters are selected. The sequence comparison algorithm then calculates the percent sequence identity for the designated test sequence(s) relative to the reference sequence, based on the selected program parameters.

Optimal alignment of sequences for comparison can be conducted, e.g., by the local homology algorithm of Smith & Waterman (1981) Adv Appl Math 2:482, by the homology alignment algorithm of Needleman & Wunsch (1970) J Mol Biol 48:443, by the search for similarity method of Pearson & Lipman (1988) Proc Natl Acad Sci USA 85:2444-2448, by computerized implementations of these algorithms (GAP, BESTFIT, FASTA, and TFASTA in the Wisconsin Genetics Software Package, Genetics Computer Group, Madison, Wis.), or by visual inspection. See generally, Ausubel et al. (1992) Current Protocols in Molecular Biology, John Wylie & Sons, Inc., New York, N.Y.

A preferred algorithm for determining percent sequence identity and sequence similarity is the BLAST algorithm, which is described in Altschul et al. (1990) J Mol Biol 215: 403-410. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hifs act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength W=11, an expectation E=10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See Henikoff & Henikoff (1989) Proc Natl Acad Sci USA 89:10915.

In addition to calculating percent sequence identity, the BLAST algorithm also performs a statistical analysis of the similarity between two sequences. See, e.g., Karlin & Altschul (1993) Proc Natl Acad Sci USA 90:5873-5887. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

I.D. Antibodies

Also provided is an antibody that specifically binds an insect GPCR polypeptide of the present invention. The term “antibody” indicates an immunoglobulin protein, or functional portion thereof, including a polyclonal antibody, a monoclonal antibody, a chimeric antibody, a single chain antibody, Fab fragments, and a Fab expression library. “Functional portion” refers to the part of the protein that binds a molecule of interest. In a preferred embodiment, an antibody of the invention is a monoclonal antibody. Techniques for preparing and characterizing antibodies are known in the art. See, e.g., Harlow & Lane (1988) Antibodies: A Laboratory Manual, Cold Spring Harbor Laboratory Press, Cold Spring Harbor, N.Y. A monoclonal antibody of the present invention can be readily prepared through use of well-known techniques such as the hybridoma techniques exemplified in U.S. Pat. No. 4,196,265 and the phage-displayed techniques disclosed in U.S. Pat. No. 5,260,203.

The phrase “specifically (or selectively) binds to an antibody”, or “specifically (or selectively) immunoreactive with”, when referring to a protein or peptide, refers to a binding reaction which is determinative of the presence of the protein in a heterogeneous population of proteins and other biological materials. Thus, under designated immunoassay conditions, the specified antibodies bind to a particular protein and do not show significant binding to other proteins present in the sample. Specific binding to an antibody under such conditions can require an antibody that is selected based on its specificity for a particular protein. For example, antibodies raised to a protein with an amino acid sequence encoded by any of the nucleic acid sequences of the invention can be selected to obtain antibodies specifically immunoreactive with that protein and not with unrelated proteins.

The use of a molecular cloning approach to generate antibodies, particularly monoclonal antibodies, and more particularly single chain monoclonal antibodies, are also provided. The production of single chain antibodies has been described in the art. See, e.g., U.S. Pat. No. 5,260,203. For this approach, combinatorial immunoglobulin phagemid libraries are prepared from RNA isolated from the spleen of the immunized animal, and phagemids expressing appropriate antibodies are selected by panning on tissue that expresses the polypeptide. The advantages of this approach over conventional hybridoma techniques are that approximately 10⁴ times as many antibodies can be produced and screened in a single round, and that new specificities are generated by heavy (H) and light (L) chain combinations in a single chain, which further increases the chance of finding appropriate antibodies. Thus, an antibody of the present invention, or a “derivative” of an antibody of the present invention, pertains to a single polypeptide chain binding molecule which has binding specificity and affinity substantially identical to the binding specificity and affinity of the light and heavy chain aggregate variable region of an antibody described herein.

The term “immunochemical reaction”, as used herein, refers to any of a variety of immunoassay formats used to detect antibodies specifically bound to a particular protein, including but not limited to competitive and non-competitive assay systems using techniques such as radioimmunoassays, ELISA (enzyme linked immunosorbent assay), “sandwich” immunoassays, immunoradiometric assays, gel diffusion precipitation reactions, immunodiffusion assays, in situ immunoassays (e.g., using colloidal gold, enzyme or radioisotope labels), Western blot analysis, precipitation reactions, agglutination assays (e.g., gel agglutination assays, hemagglutination assays), complement fixation assays, immunofluorescence assays, protein A assays, and immunoelectrophoresis assays, etc. See Harlow & Lane (1988) Antibodies: A Laboratory Manual, Cold Spring Harbor Laboratory Press, Cold Spring Harbor, N.Y. for a description of immunoassay formats and conditions.

I.E. Modulating Compounds

The term “agonist” is used throughout this application to indicate any peptide or non-peptidyl compound which increases the activity of any of the receptors of the subject invention. The term “antagonist” is used throughout this application to indicate any peptide or non-peptidyl compound which decreases the activity of any of the receptors of the subject invention.

I.F. Transgenic Organisms

It is also within the scope of the present invention to prepare a transgenic organism to express a transgene comprising nucleic acid sequences of the present invention. The term “transgenic organism”, indicates an organism comprising a germline insertion of a heterologous nucleic acid. A transgenic organism can be an animal or a plant. Transgenic organisms of the present invention are understood to encompass not only the end product of a transformation method, but also transgenic progeny thereof.

The term “transgene”, as used herein indicates a heterologous nucleic acid molecule that has been transformed into a host cell. For intended use in the creation of a transgenic organism, the transgene can include genomic sequences of the host organism at a selected locus or site of transgene integration to mediate a homologous recombination event. A transgene further comprises nucleic acid sequences of interest, for example a targeted modification of the gene residing within the locus, a reporter gene, or a expression cassette, each defined herein above.

II. G Protein-Coupled Receptors

II.A. Conserved Features

GPCRs can be subdivided into five different classes based on sequence homology and named according to their ligands (see www.gpcr.org). Class A GPCRs, sometimes called “Rhodopsin Like” receptors include amine receptors such as dopamine and octopamine, peptide receptors, rhodopsins, olfactory and nucleotide-like receptors among others. Class B GPCRs, known as “secretin Like” include calcitonin, diuretic hormone and secretin receptors in addition to others. Latrophilin and Methuselah are Class B receptors as well. The Class C Receptors or “metabotropic glutamate/pheromone” group includes metabotropic glutamate, extracellular calcium-sensing, and GABA-B receptors. Classes D and E are much smaller and are the “Fungal pheromone” and “cAMP receptors” groups respectively.

II.B. Identification of Novel Insect G Protein-Coupled Receptors

The present invention provides novel Drosophila, Heliothis and Manduca GPCR nucleic acid and polypeptide sequences. Preferably, a Drosophila, Heliothis or Manduca GPCR nucleic acid molecule of the present invention comprises the sequence set forth as any one of the odd numbered sequences of SEQ ID NOs:1-119; or a nucleic acid molecule that is substantially identical to any one of odd numbered sequences of SEQ ID NOs:1-119. Also preferably, a GPCR polypeptide of the present invention comprises an amino acid sequence set forth as any one of even numbered sequences of SEQ ID NOs:2-120; or a polypeptide that is substantially identical to any one of even numbered sequences of SEQ ID NOs:2-120.

To identify new Drosophila proteins, a database of predicted proteins (referred to herein as “the GeneMark database”) was assembled using the GeneMark program (Borodovsky & McIninch (1993) Computers & Chemistry 17:123-133) and template 50 kilobase genomic sequence scaffolds generated by Celera Corp. (Rockville, Md.). A second predicted protein database generated by Celera using an alternative protein prediction program was also used (referred to herein as “the Celera database”).

Eleven Class A GPCRs from vertebrate (either human or mouse) representing all sub-catagories of Class A were used to BLAST the GeneMark database and the Celera database. Twelve Class B and two Class C GPCRs were also used to BLAST the same databases. The results of the BLAST searches were examined and predicted proteins likely to be GPCRs were identified. A highly conserved region of cytoplasmic loop two was then used to generate Hidden Markov Model (HMM) profiles for Class A and Class B and this profile HMM was used to query the GeneMark and Celera databases to identify any Drosophila GPCRs that do not have a vertebrate homolog. The GeneMark and Celera protein predictions for each novel Drosophila GPCR were then BLASTED against the non-redundant set of GenBank. The prediction with the lower E-value score was identified and judged to be the prediction on which cloning would be based.

The corresponding genes were subsequently cloned as described in Example 2.

The novel Drosophila GPCR sequences were named according to the most closely related GPCR as shown in Table 2.

TABLE 2
BLAST Analysis of New Drosophila G protein-Coupled Receptors

| SEQ | | | | | | |
|---|---|---|---|---|---|---|
| ID | | Best Blast Hit | Score | | | |
| NO. | Inventor's Reference | (Accession) | (bits) | E value | Identities | Positives |
| 1-2 | Adenosine_99E 1 | A2 adenosine | 207 | 2.00E−52 | 118/321 | 177/321 |
| | | receptor-guinea | | | (36%) | (54%) |
| | | pig(I48095) | | | | |
| 3-4 | Allatostatin_3E1 | Galanin receptor- | 656 | 0 | 327/394 | 327/394 |
| | | Drosophila | | | (82%) | (82%) |
| | | (AF220216) | | | | |
| 5-6 | Gastrin_2C3 | trehalose receptor 1- | 224 | 2.00E−57 | 128/358 | 193/358 |
| | | Drosophila | | | (35%) | (53%) |
| | | (AB034204) | | | | |
| 7-8 | wormlike_4F9 | 7 transmembrane | 171 | 2.00E−41 | 105/323 | 163/323 |
| | | receptor, rhodopsin- | | | 32% | 49% |
| | | type family member | | | | |
| | | family member- | | | | |
| | | C. elegans | | | | |
| | | (NP_510455.2) | | | | |
| 9-10 | Dopamine_6D7 | dopamine receptor | 132 | 1E−29 | 108/338 | 163/338 |
| | | D1D -chicken | | | 31% | 47% |
| | | (C55886) | | | | |
| 11-12 | Lymnokinin_11D9 | G protein-coupled | 349 | 9.00E−95 | 237/697 | 346/697 |
| | | receptor 106 Mus | | | (34%) | (49%) |
| | | musculus | | | | |
| | | (NP_536716.1) | | | | |
| 13-14 | mthlike1_15A | GPCR-Drosophila | 137 | 3.00E−31 | 102/383 | 175/383 |
| | | yakuba (AF300421) | | | (26%) | (45%) |
| 15-16 | gastrin_17E3 | cholecystokinin | 186 | 4.00E−46 | 125/390 | 175/390 |
| | | B/gastrin receptor- | | | (32%) | (44%) |
| | | mouse (P56481) | | | | |
| 17-18 | CCK-X_26A1 | probable allatostatin | 103 | 4.00E−21 | 54/189 | 95/189 |
| | | receptor-2- | | | (28%) | (49%) |
| | | Drosophila (JC7319) | | | | |
| 19-20 | neuroYY_26B1 | neuropeptide | 60.8 | 3.00E−08 | 39/114 | 65/114 |
| | | Y/peptide YY | | | (34%) | (56%) |
| | | receptor Yc-D. rerio | | | | |
| | | (AF037401) | | | | |
| 21-22 | GRHR_27A2 | GRHR-P1- | 802 | 0.00E+01 | 404/443 | 404/443 |
| | | Drosophila | | | (91%) | (91%) |
| | | (NM_058039) | | | | |
| 23-24 | Bombesin_42A10 | BRS4 Bombesin | 186 | 7.00E−46 | 119/327 | 172/327 |
| | | Receptor-Bombina | | | (36%) | (52%) |
| | | orientalis (P47751) | | | | |
| 25-26 | Latrophillin_44D4 | latrophilin-3-bovine | 246 | 1.00E−63 | 198/756 | 326/756 |
| | | (T18405) | | | (26%) | (42%) |
| 27-28 | wormlike_47E | hypothetical protein | 164 | 2.00E−39 | 111/358 | 182/358 |
| | | F57B7.1-C. elegans | | | (31%) | (50%) |
| | | (T22835) | | | | |
| 29-30 | Calcitonin_49F9 | Calcitonin receptor- | 272 | 8.00E−72 | 148/371 | 209/371 |
| | | like-human | | | (39%) | (55%) |
| | | (XM_002673) | | | | |
| 31-32 | DiureticHor2_51A1 | Diuretic Hormone | 352 | 1E−95 | 184/386 | 239/386 |
| | | Receptor-Acheta | | | (47%) | (61%) |
| | | (Q16983) | | | | |
| 33-34 | mthlike3_54B16 | methuselah- | 605 | 1.00E−172 | 294/512 | 377/512 |
| | | Drosophila yakuba | | | (57%) | (73%) |
| | | (AF280583) | | | | |
| 35-36 | bombesin_54D4 | bombesin/gastrin- | 207 | 2.00E−52 | 126/352 | 192/352 |
| | | releasing peptide | | | (35%) | (53%) |
| | | receptor-mouse | | | | |
| | | (A39003) | | | | |
| 37-38 | 5-HT1A_56A | 5-Hydroxytryptamine | 1254 | 0.00E+01 | 648/816 | 651/816 |
| | | receptor 2A- | | | (79%) | (79%) |
| | | Drosophila (S19155) | | | | |
| 39-40 | mAcR-60C | muscarinic | 1205 | 0.00E+01 | 607/788 | 607/788 |
| | | acetylcholine | | | (77%) | (77%) |
| | | receptor-Drosophila | | | | |
| | | (S05661) | | | | |
| 41-42 | Adrenergic_60D1 | trace amine receptor | 76.6 | 8.00E−13 | 46/174 | 84/174 |
| | | 1-rat (AF380186) | | | (26%) | (47%) |
| 43-44 | mthlike9_61C | GPCR-Drosophila | 159 | 5.00E−38 | 107/431 | 193/431 |
| | | melanogaster | | | (24%) | (43%) |
| | | (AF300364) | | | | |
| 45-46 | mthlike10_61C1 | GPCR-Drosophila | 1009 | 0 | 493/533 | 500/533 |
| | | melanogaster | | | (92%) | (93%) |
| | | (AF300364) | | | | |
| 47-48 | wormlike_62 | hypothetical protein | 173 | 5.00E−42 | 121/409 | 188/409 |
| | | F57B7.1-C. elegans | | | (29%) | (45%) |
| | | (T22835) | | | | |
| 49-50 | wormlike_63A3 | hypothetical protein | 138 | 2.00E−31 | 89/275 | 143/275 |
| | | T19F4.1b-C. elegans | | | (32%) | (51%) |
| | | (U55371) | | | | |
| 51-52 | adrenergic_64A | hypothetical protein | 189 | 4.00E−47 | 105/309 | 175/309 |
| | | F59D12.1-C. elegans | | | (33%) | (55%) |
| | | (T22996) | | | | |
| 53-54 | lymnokinin_64D3 | CG10626-PA | 1011 | 0 | 501/540 | 501/540 |
| | | [Drosophila | | | (92%) | (92%) |
| | | melanogaster] | | | | |
| | | (NP_647968.2) | | | | |
| 55-56 | mthlike2_64D4 | methuselah- | 630 | 1.00E−180 | 299/501 | 366/501 |
| | | Drosophila simulans | | | (59%) | (72%) |
| | | (AF280597) | | | | |
| 57-58 | GRHR_69B | putative corazonin | 1055 | 0 | 532/579 | 534/579 |
| | | receptor [Drosophila | | | (91%) | (91%) |
| | | melanogaster] | | | | |
| | | (AAN10045.1) | | | | |
| 59-60 | somatostatin_75C1 | allatostatin | 785 | 0 | 394/467 | 394/467 |
| | | C/drostatin C | | | (84%) | (84%) |
| | | receptor 1 | | | | |
| | | [Drosophila | | | | |
| | | melanogaster] | | | | |
| | | (AAG54080.1) | | | | |
| 61-62 | NeuroYlike_77A1 | putative | 210 | 3.00E−53 | 126/356 | 174/356 |
| | | neuropeptide | | | (35%) | (48%), |
| | | receptor NPR1- | | | | |
| | | Girardia (AF329279) | | | | |
| 63-64 | Secretagogue_79A | neuromedin U | 200 | 3.00E−50 | 116/328 | 188/328 |
| | | receptor type 2- | | | (35%), | (56%), |
| | | mouse (AY057384) | | | | |
| 65-66 | 5-HT2_82C4 | serotonin receptor 5- | 1338 | 0.00E+01 | 682/868 | 683/868 |
| | | HT2-Drosophila | | | (78%), | (78%), |
| | | (X81835) | | | | |
| 67-68 | NeuroYYlike_83 | neuropeptide F | 874 | 0.00E+01 | 442/481 | 443/481 |
| | | receptor-Drosophila | | | (91%), | (91%) |
| | | (AF364400) | | | | |
| 69-70 | mAcChR_84E8 | G protein-linked | 200 | 7.00E−50 | 98/193 | 133/193 |
| | | cetylcholine receptor | | | (50%), | (68%), |
| | | GAR-2c-C. elegans | | | | |
| | | (AF272738) | | | | |
| 71-72 | Takr-86C_86C3 | tachykinin receptor | 987 | 0.00E+01 | 492/504 | 493/504 |
| | | NKD-Drosophila | | | (97%) | (97%) |
| | | (A41783) | | | | |
| 73-74 | mthlike5_87A | methuselah- | 72 | 1.00E−11 | 57/251 | 111/251 |
| | | Drosophila simulans | | | (22%) | (43%) |
| | | (AF280601) | | | | |
| 75-76 | GrowthHormone_87F1 | putative PRXamide | 986 | 0 | 508/595 | 508/595 |
| | | receptor [Drosophila | | | (85%) | (85%) |
| | | melanogaster] | | | | |
| | | (AAN10043.1) | | | | |
| 77-78 | neurotensin_88B1 | putative PRXamide | 783 | 0 | 400/430 | 400/430 |
| | | receptor [Drosophila | | | (93%) | (93%) |
| | | melanogaster] | | | | |
| | | (AAN10044.1) | | | | |
| 79-80 | DopR_88B1 | dopamine receptor | 912 | 0.00E+01 | 453/511 | 454/511 |
| | | protein 1-Drosophila | | | (88%) | (88%) |
| | | (P41596) | | | | |
| 81-82 | Octopamine_90C2 | putative octopamine | 114 | 3.00E−24 | 66/187 | 94/187 |
| | | receptor-Mamestra | | | (35%) | (49%) |
| | | (AF343878) | | | | |
| 83-84 | FSHlike_96E5 | G protein coupled | 365 | 2E−99 | 240/720 | 366/720 |
| | | receptor affecting | | | (33%) | (50%) |
| | | testicular descent - | | | | |
| | | Homo | | | | |
| | | (NP_570718.1) | | | | |
| 85-86 | Histamine_97B | histamine H2 | 52 | 2.00E−05 | 38/117 | 50/117 |
| | | receptor-dog | | | (32%) | (42%) |
| | | (A39008) | | | | |
| 87-88 | NeuropepYR_97E1 | neuropeptide Y | 931 | 0.00E+01 | 445/449 | 447/449 |
| | | receptor-Drosophila | | | (99%) | (99%) |
| | | (A41738) | | | | |
| 89-90 | Galanin_98E2 | allatostatin GPCR- | 600 | 1.00E−171 | 308/357 | 308/357 |
| | | Drosophila | | | (86%) | (86%) |
| | | (AF253526) | | | | |
| 91-92 | Takr-99D_99D1 | tachykinin receptor | 919 | 0.00E+01 | 452/519 | 453/519 |
| | | homolog DTKR- | | | (87%) | (87%) |
| | | Drosophila (S17783) | | | | |
| 93-94 | 5-HT7_100A2 | 5HT-dro serotonin | 885 | 0.00E+01 | 454/564 | 454/564 |
| | | receptor-Drosophila | | | (80%) | (80%) |
| | | (M55533) | | | | |
| 95-96 | He6Receptor_100B1 | Me6 receptor splice | 104 | 7E−21 | 107/439 | 179/439 |
| | | variant d3 Mus | | | (24%) | (40%) |
| | | musculus | | | | |
| | | (AAN33059.1) | | | | |
| 97-98 | Fsh_90c2 | FSH-TSH | 1590 | 0 | 787/831 | 787/831 |
| | | [Drosophila | | | 94% | 94% |
| | | melanogaster] | | | | |
| | | (AAB07030.1) | | | | |
| 99-100 | He6rec2_100b2 | HE6 heptahelical | 80.5 | 1.00E−13 | 104/482 | 178/482 |
| | | receptor splice | | | 21% | 36% |
| | | variant 23-Homo | | | | |
| | | sapiens | | | | |
| | | (AAN38974.1) | | | | |
| 101-102 | Prostaglandin_74f1 | prostaglandin E | 62.4 | 1.00E−08 | 61/255 | 102/255 |
| | | receptor 1 (subtype | | | 23% | 39% |
| | | EP1), 42 kDa; | | | | |
| | | Prostaglandin | | | | |
| | | E(NP_000946.1) | | | | |
| 103-104 | Oct-TyrR_79D1 | tyramine receptor - | 977 | 0 | 498/601 | 499/601 |
| | | fruit fly (Drosophila | | | 82% | 82% |
| | | sp.) (S12004) | | | | |

I. C. Identification of Heliothis G protein-coupled Receptors Heliothis virescens (hereinafter “Heliothis”) GPCR sequences were obtained by PCR using degenerate primers designed according to 5 Drosophila GPCR sequences, as described in Example 3. Heliothis GPCR fragments derived from this method were assembled by recognition of overlapping sequence. The Heliothis GPCR was named based on the most closely related insect GPCR (Table 3).

TABLE 3
BLAST Results of Heliothis GPCR

| SEQ ID | Inventor's | Best Blast Hit | Score | | | |
|---|---|---|---|---|---|---|
| NO. | Reference | (Accession) | (bits) | E value | Identities | Positives |
| 105-106 | Heliothis | GPCR-Balanus ampitrite | 407 | 1.00E−112 | 217/446 | 272/446 |
| | Oct2B | (Q93126) | | | (48%) | (60%) |

II. D. Identification of Manduca Sexta GPCR

The Diuretic Hormone GPCR nucleic acid and protein described in the application was identified and cloned by J. D. Reagan as described in J. Biol. Chem. 269 (1):9-12 (1994) (GenBank Accession No. U03489, hereby both incorporated by reference). The BLAST results are set forth below in Table 4.

TABLE 4
BLAST Analysis of Manduca GPCR

| SEQ ID | Inventor's | Best Blast Hit | Score | E | | |
|---|---|---|---|---|---|---|
| NO. | Reference | (Accession) | (bits) | value | Identities | Positives |
| 107-108 | Manduca | Diuretic Hormone | 364 | 1.00E−100 | 187/379 | 237/379 |
| | DHR | Receptor-Acheta | | | (49%) | (62%) |
| | | (Q16983) | | | | |

III. Functional Analysis of G Protein-Coupled Receptors

Many insect pests inflict plant damage by the feeding activity during larval stages. Therefore, functional analyses to assess phenotypes associated with modulation of a GPCRs during larval development can be used to identify candidate insecticide targets. The present invention discloses GPCRs whose regulation is relevant to larval viability. Thus, modulators that alter GPCR activity in a manner analogous to the changes resulting from genetic manipulations described herein below, can be useful as insecticide compositions.

III.A. Loss-of-Function Analyses

RNA-mediated interference (RNAi) is a recently discovered method to determine gene function in a number of organisms, wherein double-stranded RNA (dsRNA) directs gene-specific, post-transcriptional silencing. See, e.g., Kuwabara & Olson (2000) Parasitol Today 16(8):347-349; Bass (2000) Cell 101(3):235-238; Hunter (2000) Curr Biol 10(4):R137-140; Bosher & Labouesse (2000) Nat Cell Biol 2(2):E31-36; Sharp (1999) Genes Dev 13(2):139-141. The double-stranded RNA molecule can be synthesized in vitro and then introduced into the organism by injection or other methods. Alternatively, a heritable transgene exhibiting dyad symmetry can provide a transcript that folds as a hairpin structure. Methods for examining gene functions using dsRNAi in Drosophila are disclosed in Example 4 and further in Kennerdell & Carthew (2000) Nat Biotech 18(8):896-898; Lam & Thummel (2000) Curr Biol 10(16):957-963; Misquitta & Paterson (1999) Proc Natl Acad Sci USA 96 (4):1451-1456.

The present invention discloses RNA-mediated interference of Drosophila GPCRs Double-stranded RNA complementary to each GPCR sequence was synthesized in vitro and injected into early Drosophila embryos, as described in Example 4. Development of injected embryos was assessed by scoring: (a) morphological criteria using a light microscope (Campos-Ortega & Hartenstein (1985) The Embryonic Development of Drosophila melanogaster, Springer-Verlag, Berlin), (b) embryo hatching to become a larvae, (c) puparium formation, and (d) eclosion of the pupae as an adult fly, as indicated in Table 5 herein below. Buffer-injected embryos were injected and monitored in parallel as a control. The percentage of embryos injected with dsRNA that survive to the adult stage is set forth in

TABLE 5
Results of dsRNAi Analysis

| | | | | | | % viable |
|---|---|---|---|---|---|---|
| | | # eggs | | | | adults |
| | | showing | # | | | from |
| | # eggs | morphological | hatched | # | # | developed |
| dsRNA injected | injected | development | larvae | pupae | adults | eggs |
| none, buffer only | 941 | 806 | 580 | 500 | 433 | 0.53722 |
| ds-latrophillin 44d4 | 178 | 142 | 106 | 45 | 35 | 0.2464788 |
| Ds-5-HT2_82C4 | 186 | 153 | 56 | 46 | 39 | 0.2549019 |
| Ds-adenosine_99E1 | 169 | 136 | 70 | 49 | 37 | 0.2720588 |
| ds-mth-like 10_61C1 | 208 | 175 | 92 | 68 | 58 | 0.3314285 |
| ds-takr 99D_99D1 | 697 | 599 | 332 | 234 | 218 | 0.363939 |
| mAcR_60C | 104 | 92 | 48 | 37 | 34 | 0.3695652 |
| ds-dopr_88B1 | 108 | 80 | 44 | 37 | 30 | 0.375 |
| ds-npep YY-like 83 | 112 | 96 | 66 | 44 | 40 | 0.4166666 |
| ds-takr86c_86C3 | 107 | 91 | 61 | 44 | 38 | 0.4175824 |
| ds-gastrin 17e3 | 107 | 93 | 61 | 43 | 39 | 0.4193548 |
| ds-mthlike1 15a | 107 | 96 | 62 | 48 | 41 | 0.4270833 |
| ds-gastrin 2c3 | 219 | 185 | 104 | 95 | 81 | 0.4378378 |
| ds-wormlike(47E) | 125 | 111 | 84 | 63 | 49 | 0.4414414 |
| 5-HT7_100A2 | 95 | 79 | 48 | 36 | 35 | 0.4430379 |
| ds-mAcChR 84e | 109 | 94 | 69 | 49 | 42 | 0.4468085 |
| ds-histamine_97B | 100 | 84 | 58 | 47 | 38 | 0.4523809 |
| ds-mth-like 9_61C | 107 | 91 | 56 | 44 | 43 | 0.4725274 |
| ds-adren 64a | 172 | 154 | 115 | 87 | 74 | 0.4805194 |
| ds-fshlike 96e5 | 100 | 91 | 63 | 48 | 44 | 0.4835164 |
| ds-allatostatin_3E1 | 195 | 156 | 104 | 81 | 78 | 0.5 |
| ds-lymnokinin 64D | 191 | 155 | 116 | 92 | 78 | 0.5032258 |
| ds-cck-x 26a1 | 298 | 249 | 158 | 133 | 126 | 0.506024 |
| ds-bombesin 42a10 | 78 | 67 | 48 | 37 | 34 | 0.5074626 |
| ds-lymnokinin 11d9 | 106 | 92 | 66 | 53 | 49 | 0.5326086 |
| ds-5-ht1a 56a | 104 | 95 | 73 | 56 | 51 | 0.5368421 |
| ds-galanin 98e2 | 93 | 86 | 59 | 55 | 47 | 0.5465116 |
| ds-he6 | 94 | 84 | 64 | 52 | 46 | 0.5476190 |
| receptor_100B1 | | | | | | |
| ds-mthlike3 54b16 | 109 | 103 | 75 | 63 | 58 | 0.5631067 |
| ds-npep Y-like 77a | 191 | 164 | 109 | 103 | 93 | 0.5670731 |
| ds-grhr 27a2 | 114 | 105 | 79 | 63 | 60 | 0.5714285 |
| ds-mthlike5 87a | 102 | 94 | 68 | 60 | 56 | 0.5957446 |
| Ds- | 113 | 85 | 58 | 58 | 51 | 0.6 |
| neurotensin_88B1 | | | | | | |
| ds-octopamine 90C2 | 100 | 85 | 67 | 60 | 51 | 0.6 |
| ds-wormlike 62 | 99 | 91 | 66 | 57 | 55 | 0.6043956 |
| ds-bombesin 54d4 | 95 | 81 | 60 | 51 | 49 | 0.6049382 |
| ds-adrenergic 60d1 | 105 | 97 | 77 | 64 | 59 | 0.6082474 |
| ds-wormlike 63a3 | 103 | 94 | 82 | 62 | 60 | 0.6382978 |
| ds-npep YY26b1 | 124 | 108 | 77 | 74 | 69 | 0.6388888 |
| ds-octopamine 90c2 | 101 | 81 | 68 | 54 | 54 | 0.6666666 |
| ds-calcitonin 49f9 | 106 | 93 | 79 | 64 | 63 | 0.6774193 |
| ds-neuropep yr 97e1 | 104 | 97 | 84 | 74 | 66 | 0.6804123 |
| ds-secretagogue 79a | 103 | 96 | 76 | 71 | 67 | 0.6979166 |
| ds-wormlike 4f9 | 110 | 100 | 83 | 72 | 70 | 0.7 |
| ds-diuretichor2 51a1 | 113 | 105 | 83 | 76 | 74 | 0.7047619 |
| ds-somatostatin 75c1 | 95 | 82 | 70 | 66 | 59 | 0.7195121 |
| ds-grhr 69b | 92 | 80 | 69 | 62 | 59 | 0.7375 |
| ds-mthlike2 64d4 | 99 | 86 | 77 | 67 | 65 | 0.7558139 |
| ds-dopamine 6d7 | 109 | 103 | 86 | 84 | 79 | 0.7669902 |

Lethality resulting from loss of GPCR function is predicted to be mimicked by provision of an antagonist substance that specifically binds a given receptor. GPCR antagonists can be identified by methods known in the art and as further disclosed in the section entitled Identification of Insect GPCR Modulators, herein below. The essentiality of GPCRs, in particular, latrophillin, disclosed herein for the first time, identifies the utility of antagonists that block or mitigate the activity of GPCRs and latrophillin as insecticides.

III.B. Gain-of-Function Analyses

Ectopic expression systems have been used to elucidate gene function when classical loss-of-function genetics is not straightforward. For example, heat-induced expression of spaghetti squash, which encodes the nonmuscle myosin II regulatory light chain, can effectively rescue the early lethality of spaghetti squash mutants, facilitating the analysis of phenotypes later in development (Edwards & Kiehart (1996) Development 122:1499). Similarly, dominant phenotypes generated by over-expressing a gene of interest have been used to address post-embryonic gene functions, particularly in cases where gene mutation results in embryonic lethality. See, e.g., Lam et al. (1999) Dev Biol 212(1):204-216; Woodard et al. (1994) Cell 79(4):607-615).

Transgenic methods for ectopic expression in Drosophila utilize promoters that drive either constitutive or regulated expression of the gene of interest. Constructs designed for ectopic expression can be prepared in a transformation vector, and are introduced into the fly genome by germ line transformation. A transgenic line is established, and ectopic expression of the gene of interest can be analyzed in a wild type or mutant genetic background.

In one embodiment, a heat shock promoter is used to temporally regulate gene expression (Lis et al. (1983) Cell 35:403; Struhl (1985) Nature 318:677; Schneuwly et al. (1987) Nature 325:816). Using this approach, the level of ectopic gene expression can be easily modulated by altering the temperature and/or duration of the heat treatment. In another embodiment, gene expression may be regulated temporally or spatially by GAL4-UAS system, essentially as described by Brand and Perrimon (1993) Development 118: 401.

Over-expression of GPCRs can reveal the role of an activated GPCR. Provision of ligand and/or apo-receptor (a receptor not bound by ligand) favors formation of the liganded receptor. Similarly, provision of excess GPCR over-expression also leads to an excess of active receptor. See, e.g., Tsai et al. (1998) in Wilson et al., eds, Williams Textbook of Endocrinology, pp. 55-94, W. B. Saunders Company, Philadelphia, Pa., and references cited therein. To create this situation in vivo, a GPCR is over-expressed using a heterologous transgene. This strategy enables a functional assessment of orphan GPCRs, wherein a ligand has not yet been identified. A phenotype observed following GPCR over-expression is predicted to also be generated by abnormally elevated levels of endogenous ligand or by administration of a GPCR agonist.

III.C. Expression Pattern Analysis of GPCRs

Identification of the particular cells in which a gene is expressed aids in the determination of function and in the realization of that gene as an insecticide target. Tissues required for larval growth or viability represent better sites of insecticide action than non-essential tissues, such as the larval imaginal discs or the optic lobes of the brain which will only have a function in the adult animal.

The tissue specific expression of GPCRs is determined by examining either the localization of the mRNA transcript or by following the expression of the protein itself. Numerous methods are available to those in the art to determine in which tissues a gene transcript is expressed including, but not limited to: reverse transcriptase polymerase chain reaction (rtPCR), Northern blots and in situ hybridization. Similarly the protein localization methods are known in the art and include, but are not limited to detection by Western blot or immunocytology.

The present invention discloses the expression pattern of insect GPCRs by in situ hybridization. Embryos and wandering third instar larvae of wild-type Drosophila melanogaster (OreR strain) are processed for in situ hybridization. Embryos are processed according to the procedure found at the Berleley Drosophila Genome Project website (http://www.fruitfly.org/about/methods/RNAinsitu.html). Larvae are dissected and processed in the same manner as embryos. Hybridization probes representing a labeled antisense strand of each gene are generated and used. After hybridization, bound probe is identified with a calorimetric substrate. The location of the signal was further characterized by further dissection and mounting the tissue for compound microscope examination. Results of expression pattern analysis are described in Example 5 and Table 6.

IV. Recombinant Expression of Insect G Protein-Coupled Receptors

For recombinant production of a protein of the invention in a host organism, a nucleotide sequence encoding the protein is inserted into an expression cassette designed for the chosen host and introduced into the host where it is recombinantly produced. The choice of the specific regulatory sequences such as promoter, signal sequence, 5′ and 3′ untranslated sequence, and enhancer appropriate for the chosen host is within the level of ordinary skill in the art. The resultant molecule, containing the individual elements linking in the proper reading frame, is inserted into a vector capable of being transformed into the host cell.

Expression constructs can be transfected into a host cell by a standard method suitable for the selected host, including electroporation, calcium phosphate precipitation, DEAE-Dextran transfection, liposome-mediated transfection, infection using a retrovirus, transposon-mediated transfer, and particle bombardment techniques. The expression cassette sequence carried in the expression construct can be stably integrated into the genome of the host or it can be present as an extrachromosomal molecule.

Suitable expression vectors and methods for recombinant production of proteins are known for host organisms such as E. Coli, yeast, and insect cells. See, e.g., Lucknow & Summers (1988) Bio/Technol 6:47. Representative methods for recombinant production of an insect GPCR in E. coli are disclosed in Example 6.

Additional suitable expression vectors are baculovirus expression vectors, e.g., those derived from the genome of Autographica californica nuclear polyhedrosis virus (AcMNPV). A preferred baculovirus/insect system is PVL1392/PVL1393 used to transfect Spodoptera frugiperda (SF9) cells in the presence of linear Autographica californica baculovirus DNA (Pharmingen of San Diego, Calif.). The resulting virus is used to infect HighFive Tricoplusia ni cells (Invitrogen, Corp. of Carlsbad, Calif.). Representative methods for recombinant production of an insect GPCR in insect cells are disclosed in Example 7.

Recombinantly produced proteins can be isolated and purified using a variety of standard techniques. The actual techniques used varies depending upon the host organism used, whether the protein is designed for secretion, and other such factors. Such techniques are known to the skilled artisan. See Ausubel et al. (1992) Current Protocols in Molecular Biology, John Wylie and Sons, Inc., New York, N.Y.

The present invention further encompasses recombinant expression of the disclosed insect GPCRs, or portion thereof, in plants, as described further herein below under the section entitled Transgenic Plants.

V. Production of Antibodies Against Insect G protein-Coupled Receptors

In another aspect, the present invention provides a method of producing an antibody immunoreactive with an insect GPCR polypeptide, the method comprising recombinantly or synthetically producing an insect GPCR polypeptide, or portion thereof, to be used as an antigen. The insect GPCR polypeptide is formulated so that it is can be used as an effective immunogen. An animal is immunized with the formulated insect GPCR polypeptide to generate an immune response in the animal. The immune response is characterized by the production of antibodies that can be collected from the blood serum of the animal. Optionally, cells producing an insect GPCR antibody can be fused with myeloma cells, whereby a monoclonal antibody can be selected. Exemplary methods for producing a monoclonal antibody that recognizes an insect GPCR protein are described in standard laboratory protocol manuals available to those of ordinary skill in the art. Preferred embodiments of the method use a polypeptide set forth as any one of even numbered sequences of SEQ ID NOs:2-120.

The present invention also encompasses antibodies and cell lines that produce monoclonal antibodies as described herein.

The foregoing antibodies can be used in methods known in the art relating to the localization and activity of the insect GPCR polypeptide sequences of the invention, e.g., for cloning of insect GPCR nucleic acids, immunopurification of insect GPCR polypeptides, imaging insect GPCR polypeptides in a biological sample, and measuring levels thereof in appropriate biological samples.

VI. Methods for Detecting an Insect Receptor Nucleic Acid

In another aspect of the invention, a method is provided for detecting a nucleic acid molecule that encodes an insect GPCR polypeptide. Such methods can be used to detect insect GPCR gene variants and related resistance gene sequences. The disclosed methods facilitate genotyping, cloning, gene mapping, and gene expression studies.

The nucleic acids of the present invention can be used to clone genes and genomic DNA comprising the disclosed sequences. Alternatively, the nucleic acids of the present invention can be used to clone genes and genomic DNA of related sequences, preferably GPCR genes in pest insects and nematodes. Using the nucleic acid sequences disclosed herein, such methods are known to one skilled in the art. See, for example, Sambrook et al., eds (1989) Molecular Cloning, Cold Spring Harbor Laboratory Press, Cold Spring Harbor, N.Y. Representative methods are also disclosed in Examples 2 and 3. Preferably, the nucleic acids used for this method comprise sequences set forth as any one of odd numbered sequences of SEQ ID NOs:1-107.

In one embodiment, genetic assays based on nucleic acid molecules of the present invention can be used to screen for genetic variants by a number of PCR-based techniques, including single-strand conformation polymorphism (SSCP) analysis (Orita et al. (1989) Proc Natl Acad Sci USA 86(8):2766-2770), SSCP/heteroduplex analysis, enzyme mismatch cleavage, direct sequence analysis of amplified exons (Kestila et al. (1998) Mol Cell 1(4):575-582; Yuan et al. (1999) Hum Mutat 14(5):440-446), allele-specific hybridization (Stoneking et al. (1991) Am J Hum Genet 48(2):370-82), and restriction analysis of amplified genomic DNA containing the specific mutation. Automated methods can also be applied to large-scale characterization of single nucleotide polymorphisms (Brookes (1999) Gene 234(2):177-186; Wang et al. (1998) Science 280(5366):1077-1082). Preferred detection methods are non-electrophoretic, including, for example, the TaqMan™ allelic discrimination assay, PCR-OLA, molecular beacons, padlock probes, and well fluorescence. See Landegren et al. (1998) Genome Res 8:769-776.

VII. Methods for Detecting an Insect G Protein-Coupled Receptor Polypeptide

In another aspect of the invention, a method is provided for detecting a level of insect GPCR polypeptide using an antibody that specifically recognizes an insect GPCR polypeptide, or portion thereof. In a preferred embodiment, biological samples from an experimental subject and a control subject are obtained, and insect GPCR polypeptide is detected in each sample by immunochemical reaction with the insect GPCR antibody. More preferably, the antibody recognizes amino acids of any one of even numbered sequences of SEQ ID NOs:2-120; and is prepared according to a method of the present invention for producing such an antibody.

In one embodiment, an insect GPCR antibody is used to screen a biological sample for the presence of an insect GPCR polypeptide. A biological sample to be screened can be a biological fluid such as extracellular or intracellular fluid, or a cell or tissue extract or homogenate. A biological sample can also be an isolated cell (e.g., in culture) or a collection of cells such as in a tissue sample or histology sample. A tissue sample can be suspended in a liquid medium or fixed onto a solid support such as a microscope slide. In accordance with a screening assay method, a biological sample is exposed to an antibody immunoreactive with an insect GPCR polypeptide whose presence is being assayed, and the formation of antibody-polypeptide complexes is detected. Techniques for detecting such antibody-antigen conjugates or complexes are known in the art and include but are not limited to centrifugation, affinity chromatography and the like, and binding of a labeled secondary antibody to the antibody-candidate receptor complex.

A modulator that shows specific binding to an insect modulator can also be used to detect an insect GPCR. Representative techniques for assaying specific binding include are described herein above under the heading “Identification of Insect GPCR Modulators”.

The disclosed methods for detecting an insect GPCR polypeptide can be useful to determine altered levels of gene expression that are associated with particular conditions such as enhanced tolerance to insecticides that target a particular insect GPCR polypeptide.

VIII. Identification of GPCR Modulators

The present invention further discloses a method for identifying a compound that modulates an insect GPCR. As used herein, the terms “candidate substance” and “candidate compound” are used interchangeably and refer to a substance that is believed to interact with another moiety, wherein a biological activity is modulated. For example, a representative candidate compound is believed to interact with an insect GPCR polypeptide, or fragment thereof, and can be subsequently evaluated for such an interaction. Exemplary candidate compounds that can be investigated using the methods of the present invention include, but are not restricted to, viral epitopes, peptides, enzymes, enzyme substrates, co-factors, lectins, sugars, oligonucleotides or nucleic acids, oligosaccharides, proteins, chemical compounds, small molecules, and antibodies. A candidate compound to be tested can be a purified molecule, a homogenous sample, or a mixture of molecules or compounds.

As used herein, the term “modulate” means an increase, decrease, or other alteration of any or all chemical and biological activities or properties of a wild-type insect GPCR polypeptide, preferably an insect GPCR polypeptide of any one of the even-numbered SEQ ID NOs:2-108. Preferably, an insect GPCR modulator is an agonist of an insect GPCR protein activity.

In accordance with the present invention there is also provided a rapid and high throughput screening method that relies on the methods described above. This screening method comprises separately contacting each compound with a plurality of substantially identical target molecules. In such a screening method the plurality of target molecules preferably comprises more than about 10⁴ samples, or more preferably comprises more than about 5×10⁴ samples. In an alternative high-throughput strategy, each target molecule can be contacted with a plurality of candidate compounds.

In one embodiment, the disclosed methods for identifying modulators of insect GPCRs are performed using GPCR sequences set forth as any one of even-numbered SEQ ID NOS:2-108. In particular, the loss-of-function lethality that is observed when latrophilin function is disrupted which suggests that antagonists of latrophilin can be useful as insecticides.

The disclosed methods for identifying modulators of insect GPCRs can be performed using nucleic acid sequences derived from a pest organism. The GPCR sequences disclosed herein provide methods for identifying homologous sequences in pest species. Such techniques are well know to those in the art. See, for example, Sambrook et al., eds (1989) Molecular Cloning, Cold Spring Harbor Laboratory Press, Cold Spring Harbor, N.Y., and Examples 2 and 3 herein below.

In a preferred embodiment, the disclosed methods for identifying modulators employ a Heliothis octopamine GPCR polypeptide.

Representative methods for identification of a substance that binds and thereby modulates an insect GPCR are disclosed herein below. The term “binding” refers to an affinity between two molecules, for example, a ligand and a receptor. As used herein, “binding” means a preferential binding of one molecule for another in a mixture of molecules. The binding of the molecules can be considered specific if the binding affinity is about 1×10⁴ M⁻¹ to about 1×10⁶ M⁻¹ or greater. Binding of two molecules also encompasses a quality or state of mutual action such that an activity of one protein or compound on another protein is inhibitory (in the case of an antagonist) or enhancing (in the case of an agonist). To demonstrate saturable binding of a candidate compound, identified by any such method, to a GPCR ligand binding domain, Scatchard analysis can be carried out as described, for example, by Mak et al. (1989) J Biol Chem 264:21613:21618.

VIII.A. Protein Binding Assays

Several techniques can be used to detect interactions between a protein and a chemical ligand without employing an in vivo ligand. Representative methods include, but are not limited to, Fluorescence Correlation Spectroscopy, Surface-Enhanced Laser Desorption/Ionization Time-Of-flight Spectroscopy, and Biacore technology, as described herein below. These methods are amenable to automated, high-throughput screening.

Fluorescence Correlation Spectroscopy (FCS) measures the average diffusion rate of a fluorescent molecule within a small sample volume (Madge et al. (1972) Phys Rev Lett 29:705-708; Maiti et al. (1997) Proc Natl Acad Sci USA 94:11753-11757). The sample size can be as low as 10³ fluorescent molecules and the sample volume as low as the cytoplasm of a single bacterium. The diffusion rate is a function of the mass of the molecule and decreases as the mass increases. FCS can therefore be applied to polypeptide-ligand interaction analysis by measuring the change in mass and therefore in diffusion rate of a molecule upon binding. In a typical experiment, the target to be analyzed is expressed as a recombinant polypeptide with a sequence tag, such as a poly-histidine sequence, inserted at the N-terminus or C-terminus. The expression takes place in E. coli, yeast or mammalian cells. The polypeptide is purified using chromatographic methods. For example, the poly-histidine tag can be used to bind the expressed polypeptide to a metal chelate column such as Ni²⁺ chelated on iminodiacetic acid agarose. The polypeptide is then labeled with a fluorescent tag such as carboxytetramethylrhodamine or BODIPY™ (Molecular Probes of Eugene, Oreg.). The polypeptide is then exposed in solution to the potential ligand, and its diffusion rate is determined by FCS using instrumentation available from Carl Zeiss, Inc. (Thornwood, New York). Ligand binding is determined by changes in the diffusion rate of the polypeptide.

Surface-Enhanced Laser Desorption/lonization (SELDI) was developed by Hutchens & Yip (1993) Rapid Commun Mass Spectrom 7:576-580. When coupled to a time-of-flight mass spectrometer (TOF), SELDI provides a technique to rapidly analyze molecules retained on a chip. It can be applied to ligand-protein interaction analysis by covalently binding the target protein, or portion thereof, on the chip and analyzing by MS the small molecules that bind to this protein (Worrall et al. (1998) Anal Biochem 70:750-756). In a typical experiment, the target to be analyzed is expressed as described for FCS. The purified protein is then used in the assay without further preparation. It is bound to the SELDI chip either by utilizing the poly-histidine tag or by other interaction such as ion exchange or hydrophobic interaction. The chip thus prepared is then exposed to the potential ligand via, for example, a delivery system able to pipet the ligands in a sequential manner (autosampler). The chip is then washed in solutions of increasing stringency, for example a series of washes with buffer solutions containing an increasing ionic strength. After each wash, the bound material is analyzed by submitting the chip to SELDI-TOF. Ligands that specifically bind the target are identified by the stringency of the wash needed to elute them.

Biacore relies on changes in the refractive index at the surface layer upon binding of a ligand to a target polypeptide immobilized on the layer. In this system, a collection of small ligands is injected sequentially in a 2-5 microliter cell, wherein the target polypeptide is immobilized within the cell. Binding is detected by surface plasmon resonance (SPR) by recording laser light refracting from the surface. In general, the refractive index change for a given change of mass concentration at the surface layer is practically the same for all proteins and peptides, allowing a single method to be applicable for any protein (Liedberg et al. (1983) Sensors Actuators 4:299-304; Malmquist (1993) Nature 361:186-187). In a typical experiment, the target to be analyzed is expressed as described for FCS. The purified protein is then used in the assay without further preparation. It is bound to the Biacore chip either by utilizing the poly-histidine tag or by other interaction such as ion exchange or hydrophobic interaction. The chip thus prepared is then exposed to the potential ligand via the delivery system incorporated in the instruments sold by Biacore (Uppsala, Sweden) to pipet the ligands in a sequential manner (autosampler). The SPR signal on the chip is recorded and changes in the refractive index indicate an interaction between the immobilized target and the ligand. Analysis of the signal kinetics of on rate and off rate allows the discrimination between non-specific and specific interaction.

VIII.B. Peptide Interaction Assays

Methods for displaying diverse peptide libraries enable rapid library construction, amplification, and selection of ligands directed against a target molecule. See Lowman (1997) Annu Rev Biophys Biomol Struct 26:401-424; Sidhu (2000) Curr Opin Biotech 11(6):610-616; and U.S. Pat. No. 5,510,240. Assays can also be employed that select peptides capable of disrupting the interaction between a GPCR and a requisite co-factor, as described by Hall et al. (2000) Mol Enodcrinol 14(12):2010-2023; Northrop et al. (2000) Mol Endocrinol 14(5)605-622; International Publication No. WO 00/37077, herein incorporated by reference.

VIII.C. Transcriptional Assays

The present invention also provides methods for identifying modulators of insect GPCR transcriptional activation. One strategy employs an expression system comprising: (1) an insect GPCR comprising a functional ligand binding domain of an insect GPCR, (2) a target gene expression cassette comprising a response element regulated by the GPCR operatively linked to a reporter gene, and (3) a test compound. Methods for constructing a GPCR gene and a target gene expression cassette are described herein below under the heading “Chimeric Receptors for Inducible Gene Expression”. See also, Wentworth et al. (2000) J Endocrinol 166(3):R11-16; Yang & Chen (1999) Cancer Res 59(18):4519-4524, and U.S. Pat. No. 4,981,784, herein incorporated by reference.

The term “reporter gene” refers to a heterologous gene encoding a product that is readily observed and/or quantitated. A reporter gene is heterologous in that it originates from a source foreign to an intended host cell or, if from the same source, is modified from its original form. Any suitable reporter and detection method can be used in accordance with the disclosed methods. Non-limiting examples of detectable reporter genes that can be operatively linked to a transcriptional regulatory region can be found in Alam and Cook (1990) Anal Biochem 188:245-254 and International Publication No. WO 97/47763. Preferred reporter genes for transcriptional analyses include the lacZ gene (See, e.g., Rose and Botstein (1983) Meth Enzymol 101:167-180), Green Fluorescent Protein (GFP) (Cubitt et al. (1995) Trends Biochem Sci 20:448455), luciferase, or chloramphenicol acetyl transferase (CAT).

An amount of reporter gene can be assayed by any method for qualitatively, or preferably quantitatively, determining presence or activity of the reporter gene product. The amount of reporter gene expression directed by each test substance is compared to an amount of reporter gene expression in the absence of a test substance. A test substance is identified as having agonist activity when there is significant increase in a level of reporter gene expression in the presence of the substance when compared to a level of reporter gene expression in the absence of the test substance. The term “significant increase”, as used herein, refers to an quantified change in a measurable quality that is larger than the margin of error inherent in the measurement technique, preferably an increase by about 2-fold or greater relative to a control measurement, more preferably an increase by about 5-fold or greater, and most preferably an increase by about 10-fold or greater.

VIII.D. Rational Design

The knowledge of the structure a native GPCR polypeptide provides an approach for rational pesticide design. See, e.g. Schapira et al. (2000) Proc Natl Acad Sci USA 97(3):1008-1013. The structure of a GPCR polypeptide can be determined by X-ray crystallography or by computational algorithms that generate three-dimensional representations. See Huang et al. (2000) Pac Symp Biocomput 230-41; Saqi et al. (1999) Bioinformatics 15:521-522; International Publication No. WO 99/26966, herein incorporated by reference. Alternatively, a working model of a GPCR structure can be derived by homology modeling (Huang et al. (2000) Acta Pharmacol Sin. Jun;21(6):529-35. Computer models can further predict binding of a protein structure to various substrate molecules that can be synthesized and tested. Additional compound design techniques are described in U.S. Pat. Nos. 5,834,228 and 5,872,011.

IX. Methods for Pest Control

Another aspect of the present invention is a method for pest control by modulation of insect GPCR biological activity. Substances having such activity can be discovered by the methods disclosed herein and include, but are not limited to, chemical compounds, antibodies, and gene products encoded by plant transgenes.

The present invention provides methods for preventing the onset or progression of a pest infestation in a plant. The method comprises administering a modulator of a GPCR set forth as any one of the even-numbered SEQ ID NOs:2-120, wherein modulation of the GPCR results in organismal lethality. Preferably, the lethality occurs during larval development.

IX.A. Formulation

An insect GPCR modulator of the present invention is typically formulated using acceptable vehicles, adjuvants, and carriers as desired. Representative formulations include emulsifiable concentrates, water-miscible liquids, wettable powders, water-soluble powders, oil solutions, flowable powders, aerosols, vapors, granulars, microcapsules, fumigants, ultra-low volume concentrates, fogging concentrates, vapors, impregnating materials, poison baits, and seed dressings. See, e.g., Perry et al. (1997) Insecticides in Agriculture and Environment: Retrospects and Prospects, pp. 7-10, Springer-Verlag, New York, N.Y. A formulation can be further selected based on its ability to improve insecticide properties such as storage, handling, application, effectiveness, safety to the applicator and the environment, and cost.

An insecticide formulation can further include a synergist that can enhance the activity of an insect GPCR modulator of the present invention. See Yamamoto (1973) in Casida, ed, Pyrethrum, The Natural Insecticide, pp.191-170, Academic Press, New York, N.Y.; Hodgson & Tate (1976) in Wilkinson, ed, Insecticide Biochemistry and Physiology, pp. 115-148, Plenum Press, New York, N.Y.; Wilkinson (1976a) in Tahori, ed, Proc 2^{nd} Int Congr on Pesticides and Chemistry, Vol. 2, pp. 117-159, Gordon & Breach, New York, N.Y.; Wilkinson (1976b) in Metcalf & McKelvey, eds, The Future for Insecticides: Needs and Prospects, Vol. 6, pp. 191-178, Wiley, New York, N.Y.; Casida & Quistad (1995) in Casida & Quistad, eds, Pyrethrum Flowers: Production, Chemistry, Toxicology, and Uses, pp. 258-276, Oxford University Press, New York, N.Y. Alternatively, synergism can be accomplished by treatment of a plant prior to application of an insect GPCR modulator, or by application of a synergist at sites on a plant distinct form sites of application of an insect GPCR modulator.

IX.B. In Vivo Assays

The insecticidal activity of a modulator of an insect GPCR can be tested using standard techniques in the art, including topical application, injection, dipping, contact or residual exposure, and feeding/drinking. See, e.g., Perry et al. (1997) Insecticides in Agriculture and Environment: Retrospects and Prospects, pp. 12-13, Springer-Verlag, New York, N.Y. As one example, a formulation comprising a modulator is sprayed on a plant, insect larvae are then applied to the plant, and after an appropriate temporal duration, a degree of plant destruction by the larvae is quantitated.

IX.C. Dose and Administration

The toxicity of an insecticide to an organism can be expressed in terms of the amount of compound per unit weight of the organism required to kill 50% of the test population, also referred to as the lethal dose (LD₅₀). The LD₅₀ is usually expressed in milligrams per kilogram of body weight or micrograms per insect. The lethal concentration (LC₅₀) is the concentration of a compound in an external medium that is required to kill 50% of the test population, and is expressed as the percentage or parts per million (ppm) of the active ingredient (Al) in the medium. This value can be used when the exact dose administered to an insect cannot be determined. The effectiveness of a candidate insecticidal substance can also be assayed in terms of lethal time (LC₅₀). LC₅₀ represents the time required to elicit 50% mortality of the test organisms at a specified dose or concentration and is a suitable measure for field tests.

In some instances, a rate of knockdown rather than lethality is measured as a criterion of effectiveness. In such cases the knockdown dose (KD₅₀) or the knockdown time (KT₅₀) can be used to express insecticidal activity.

The present invention also envisions the identification of insecticidal substances wherein killing or knockdown does not constitute the desired criterion. For example, useful assays can also assess non-lethal measures such as, for example, progression to developmental stages, fecundity, egg viability, attractant or repellant activity, paralysis, and anti-feeding activity.

Insect GPCR modulators identified in accordance with methods of the present invention are useful for preventing or treating an insect infestation, and in some cases a nematode infestation, in a plant or animal. Prevention and treatment methods employ an effective amount of the modulator. The term “effective amount” as used herein refers to an amount effective to prevent or ameliorate infestation.

An effective amount can comprise a range of amounts. One skilled in the art can readily assess the potency and efficacy of an insect GPCR modulator of the present invention and adjust the administration regimen accordingly. A modulator of insect GPCR biological activity can be evaluated by a variety of techniques, for example, by using a responsive reporter gene in an transcriptional assay, by assaying interaction of insect GPCR polypeptides with a monoclonal antibody, or by assaying insect viability when a modulator is administered to an insect, each technique described herein. One of ordinary skill in the art can tailor the dosages to a particular application, taking into account the particular formulation and method of administration to be used with the composition as well as the type of plant or animal, the development stage of the plant or animal, and the severity of the infestation to be treated.

IX.D. Transgenic Plants

The present invention also encompasses methods for pest control wherein an insect GPCR modulator is expressed in a plant. Preferably, a nucleic acid, peptide or polypeptide encoded by a transgene in a plant modulates the activity of any of SEQ ID NOs:1-119. In one embodiment, a transgene can encode a peptide that specifically binds an insect GPCR of the present invention. In another embodiment, a construct encoding an antibody that specifically binds an insect GPCR of the present invention can be expressed in plants to confer insect control. See, e.g., U.S. Pat. No. 5,686,600, the contents of which are herein fully incorporated by reference. Methods for generating a transgenic plant are known in the art and are discussed further herein below.

IX.E. Target Organisms

Insect GPCR modulators discovered according to the methods disclosed herein can be used for the prevention or amelioration of a pest infestation. The term “pest” as used herein refers to any organism that damages a plant, including mature plants, seedlings, and stored grain. The term “pest” also refers to any organism that causes disease in an animal. The compositions and methods disclosed herein are envisioned to be particularly useful to prevent or to treat infestation of insect pests, including but not limited to aphids, locusts, spider mites, boll weevils, and pests which attack stored grains (e.g., Tribolium and Tenebrio). The present disclosure is also relevant to methods for controlling soil nematodes and plant-parasitic nematodes such as Melooidogyne.

XI. Transgenic Plants

The present invention envisions expression of insect GPCR modulators, antibodies, dsRNA, antisense RNA or components of a GPCR in plants. Representative techniques for transforming dicotyledonous and monocotyledonous plants are described herein below.

The phrase “a plant, or parts thereof”, as used herein shall mean an entire plant; and shall mean the individual parts thereof, including but not limited to seeds, leaves, stems, and roots, as well as plant tissue cultures. Transgenic plants of the present invention are understood to encompass not only the end product of a transformation method, but also transgenic progeny thereof.

Representative plants that can be used in transgenic methods disclosed herein include but are not limited to rice, wheat, barley, rye, corn, potato, carrot, sweet potato, sugar beet, bean, pea, chicory, lettuce, cabbage, cauliflower, broccoli, turnip, radish, spinach, asparagus, onion, garlic, eggplant, pepper, celery, carrot, squash, pumpkin, zucchini, cucumber, apple, pear, quince, melon, plum, cherry, peach, nectarine, apricot, strawberry, grape, raspberry, blackberry, pineapple, avocado, papaya, mango, banana, tobacco, tomato, sorghum and sugarcane.

XI.A. Promoters

For in vivo production of an insect GPCR modulator or a chimeric receptor expression cassette in plants, exemplary constitutive promoters are derived from the CaMV 35S, rice actin, and maize ubiquitin genes. See Binet et al. (1991) Plant Sci 79:87-94, Christensen et al. (1989) Plant Mol Biol 12:619-632, Callis et al. (1990) J Biol Chem 265:12486-12493, Norris et al. (1993) Plant Mol Biol 21:895-906, European Patent Application Nos. 0 342 926 and 0 392 225, Taylor et al (1993) Plant Cell Rep 12:491-495, McElroy et al (1990) Plant Cell 2:163-171, McEclroy et al. (1991) Mol Gen Genet 231:150-160, Chibbar et al. (1993) Plant Cell Rep 12:506-509. Representative inducible promoters suitable for use with the present invention include the chemically inducible PR-1 promoter, the PR-1a promoter, an ethanol-inducible promoter, a glucocorticoid inducible promoter, and a wound-inducible promoter. See Uknes et al. (1992) Plant Cell 4:645-656, Lebel et al. (1998) Plant J 16:223-233, Caddik et al. (1998) Nat Biotechnol 16:177-180, Aoyama & Chua (1997) The Plant Journal 11:605-612, Xu et al. (1993) Plant Mol Biol 22:573-588; Logemann et al. (1989) Plant Cell 1:151-158, Rohrmeier & Lehle (1993) Plant Mol Biol 22:783-792, Firek et al. (1993) Plant Mol Biol 22:129-142, and Warner et al. (1993) Plant J 3:191-201. Selected promoters can direct expression in specific cell types (such as leaf epidermal cells, mesophyll cells, root cortex cells) or in specific tissues or organs (roots, leaves or flowers, for example). Representative promoters that direct cell- or tissue-specific expression in plants and can be used in accordance with the present invention include but are not limited to a root-specific promoter (de Framond (1991) FEBS 290:103-106, U.S. Pat. No. 5,466,785), a pith-preferred promoter (International Publication No. WO 93/07278), a leaf-specific promoter (Hudspeth & Grula (1989) Plant Mol Biol 12:579-589), and a pollen-specific promoter (International Publication No. WO 93/07278).

XI.B. Vectors

The expression cassette is cloned into a vector suitable for transformation. Suitable expression vectors which can be used include, but are not limited to, the following vectors or their derivatives: plant transformation vectors, viruses such as vaccinia virus or adenovirus, baculovirus vectors, yeast vectors, bacteriophage vectors (e.g., lambda phage), plasmid and cosmid DNA vectors, and transposon-mediated transformation vectors.

Numerous vectors available for plant transformation are known to those of ordinary skill in the plant transformation arts, and the genes pertinent to this invention can be used with any such vectors. Exemplary vectors include pCIB200, pCIB2001, pCIB10, pCIB3064, pSOG19, and pSOG35. The selection of vector will depend upon the preferred transformation technique and the target species for transformation.

Many vectors are available for transformation using Agrobacterium tumefaciens. These typically carry at least one T-DNA border sequence and include vectors such as pBIN19 (Bevan (1984) Nuc Acids Res 12:8711-8721) and pXYZ. See also European Patent Application No. 0 332 104, herein incorporated by reference.

Transformation without the use of Agrobacterium tumefaciens circumvents the requirement for T-DNA sequences in the chosen transformation vector and consequently vectors lacking these sequences can be utilized in addition to vectors such as the ones described above which contain T-DNA sequences. Transformation techniques that do not rely on Agrobacterium include transformation via particle bombardment, protoplast uptake (e.g., electroporation), and microinjection. The choice of vector depends largely on the preferred selection for the plant species being transformed.

For certain target species, different antibiotic or herbicide selection markers can be preferred. Selection markers used routinely in transformation include the nptII gene, which confers resistance to kanamycin and related antibiotics (Messing & Vierra (1982) Gene 19: 259-268; Bevan et al. (1983) Nature 304:184-187), the bar gene, which confers resistance to the herbicide phosphinothricin (White et al. (1990) Nuc Acids Res 18:1062, Spencer et al. (1990) Theor Appl Genet 79:625-631), the hph gene, which confers resistance to the antibiotic hygromycin (Blochlinger & Diggelmann (1984) Mol Cell Biol 4:2929-2931), and the dhfr gene, which confers resistance to methatrexate (Bourouis et al. (1983) EMBO J 2(7):1099-1104), the EPSPS gene, which confers resistance to glyphosate (U.S. Pat. Nos. 4,940,935 and 5,188,642), and the mannose-6-phosphate isomerase gene, which provides the ability to metabolize mannose (U.S. Pat. Nos. 5,767,378 and 5,994,629).

XI.C. Transformation of Dicotyledons

Transformation techniques for dicotyledons are known in the art and include Agrobacterium-based techniques and techniques that do not require Agrobacterium. Non-Agrobacterium techniques involve the uptake of exogenous genetic material directly by protoplasts or cells. This can be accomplished by polyethylene glycol (PEG) electroporation, particle bombardment-mediated uptake, or microinjection. Examples of these techniques are described by Paszkowski et al. (1984) EMBO J 3:2717-2722; Potrykus et al. (1985) Mol Gen Genet 199:169-177; Reich et al. (1986) Biotechnology 4:1001-1004; Klein et al. (1987) Nature 327:70-73; and U.S. Pat. Nos. 4,945,050, 5,036,006, and 5,100,792. Using any of the afore-mentioned methods, the transformed cells can be regenerated to whole plants using standard techniques known in the art.

XI.D. Transformation of Monocotyledons

Transformation of most monocotyledon species has now also become routine. Preferred techniques include direct gene transfer into protoplasts using PEG or electroporation techniques, and particle bombardment into callus tissue. See European Patent Application Nos. 0 292 435, 0 392 225, and 0 332 581; International Publication Nos. WO 93/07278 and WO 93/21335; Gordon-Kamm et al. (1990) Plant Cell 2:603-618; Fromm et al. (1990) Biotechnology 8:833-839; Koziel et al. (1993) Biotechnology 11:194-200; Zhang et al. (1988) Plant Cell Rep 7:379-384; Shimamoto et al. (1989) Nature 338:274-277; Datta et al. (1990) Biotechnology 8:736-740; Christou et al. (1991) Biotechnology 9:957-962; Vasil et al. (1992) Biotechnology 10:667-674; Vasil et al. (1993) Biotechnology 11:1553-1558; and Weeks et al. (1993) Plant Physiol 102:1077-1084. More recently, transformation of monocotyledons using Agrobacterium has been described. See International Publication No. WO 94/00977 and U.S. Pat. No. 5,591,616, both of which are incorporated herein by reference.

XII. Methods of Inducible Gene Expression

The present invention further provides a method of controlling gene expression in an organism, the method comprising: (a) transforming the organism with a receptor expression cassette comprising a 5′ regulatory region capable of promoting expression operatively linked to a receptor cassette encoding a chimeric receptor polypeptide of the invention, and a 3′ terminating region; (b) transforming the organism with a target expression cassette comprising a 5′ regulatory region operatively linked to a target nucleotide sequence, wherein the 5′ regulatory region comprises one or more response elements that are regulated by G protein signaling; (c) expressing the chimeric receptor polypeptide in the organism; and (d) contacting the organism with a chemical ligand that binds to the ligand binding domain of the chimeric receptor polypeptide, whereby the chimeric receptor polypeptide activates expression of the target nucleotide sequence through a G protein signaling pathway.

In a preferred embodiment, GPCR cassettes comprising disclosed GPCR sequences are useful for the regulation of expression of target polypeptides in plants in the presence of appropriate chemical ligands. For example, U.S. Pat. No. 5,880,333 is drawn to a method for controlling gene expression in plants comprising transforming a plant with expression cassette encoding a nuclear receptor polypeptide and a target sequence. The method is useful for controlling various traits of agronomic importance.

The present invention provides for the production of plants containing GPCR antagonist RNAs. These antagonistic RNAs are typically derived from plant transgenes containing antisense oriented genes or from transgenes that make mRNAs that have the ability to fold to make a hairpin structure (Patel and Jacobs-Lorena (1988) Proceedings of the National Academy of Science: 85, 9601; Zhao and Pick (1993) Nature 365, 448; Lam and Thummel (2000) Current Biology 10, 957. Insects consuming plant material containing these RNAs would have reduced viability due to a decrease in the essential GPCR transcript level.

### EXAMPLES

The following Examples have been included to illustrate modes of the invention. Certain aspects of the following Examples are described in terms of techniques and procedures found or contemplated by the present co-inventors to work well in the practice of the invention. These Examples illustrate standard laboratory practices of the co-inventors. In light of the present disclosure and the general level of skill in the art, those of skill will appreciate that the following Examples are intended to be exemplary only and that numerous changes, modifications, and alterations can be employed without departing from the scope of the invention.

### Example 1

### Database Searches

To identify new Drosophila proteins, a database of predicted proteins (referred to herein as “the GeneMark database”) was assembled using the GeneMark program (Borodovsky & McIninch (1993) Computers & Chemistry 17:123-133) and template 50 kilobase genomic sequence scaffolds generated by Celera Corp. (Rockville, Md.). A second predicted protein database generated by Celera using an alternative protein prediction program was also used (referred to herein as “the Celera database”).

Eleven Class A GPCRs from vertebrate (either human or mouse) representing all sub-catagories of Class A were used to BLAST the GeneMark database and the Celera database. Twelve Class B and two Class C GPCRs were also used to BLAST the same databases. The results of the BLAST searches were examined and predicted proteins likely to be GPCRs were identified. A highly conserved region of cytoplasmic loop two was then used to generate Hidden Markov Model (HMM) profiles for Class A and Class B according to the HMMER 2.1.1 program (available from Washington University School of Medicine, St. Louis, Mo.). HMMER 2.1.1 hmmbuild parameters were selected for maximal sensitivity. The profile HMM was further calibrated according to the program instructions. This profile HMM was used to query the GeneMark and Celera databases to identify any Drosophila GPCRs that do not have a vertebrate homolog. The GeneMark and Celera protein predictions for each novel Drosophila GPCR were then BLASTED against the non-redundant set of GenBank. The prediction with the lower E-value score was identified and judged to be the prediction on which cloning would be based.

### Example 2

### Isolation of Drosophila melanogaster GPCR cDNAs

cDNA clones of Drosophila GPCRs were cloned by PCR using a first strand Drosophila cDNA pool as template. PCR primers were designed to include the predicted start and stop codons of each receptor using the primer3 application (available through Whitehead/MIT Center for Genome Research of Cambridge, Mass.). Amplified products were cloned in the pUNI/V5-His-TOPO or pCR2.1-TOPO vectors (Invitrogen Corp. of Carlsbad, Calif.). Cloned inserts were sequenced on both strands by primer walking using an ABI PRISM® 3700 DNA Analyzer (Applied Biosystems of Foster City, Calif.) to an accuracy of <1/10,000 nucleotide errors.

### Example 3

### Cloning the Heliothis virescens Octopamine GPCR by PCR

PolyA+ RNA was made from Heliothis virescens larval gut tissue. cDNA was prepared by reverse transcription using random primers and a Gene Amp kit (Perkin Elmer of San Jose, Calif.). The reverse transcription reaction was allowed to proceed for 15 minutes at 42C, and then stopped by incubating the reaction for 5 minutes at 99C. For amplification of the octopamine receptor, nested, degenerate primers were designed according to the amino acid sequence of the Drosophila OAMB receptor and a barnacle octopamine receptor (forward degenerate primer: 5′gcatgtctagaggngaybtntggtgyhsnrtntgg 3′ (SEQ ID NO:109) and the reverse degenerate primer: 5′gcatgaagcttngtyttngcngcyttngtytccat 3′ (SEQ ID NO:110). Primers included XbaI and HindIII restriction sites for cloning into pBluescript (Stratagene of La Jolla, Calif.). Cycling parameters for amplification using degenerate primers were as follows: initial amplification for 105 seconds at 95C; 30 cycles—20 seconds at 95C, 30 seconds at 63C, 2 minutes at 72C; final amplification for 7 minutes at 72C; hold at 4C. Each sample was purified over a Qiagen PCR Purification column/kit and used as template in a second amplification step identical to the first. An approximately 1.1 kb band was identified from sequencing as the Heliothis virescens octopamine receptor.

Amplification of cDNA ends was performed using a MARATHON RACE kit (Clonetech Laboratories, Inc. of Palo Alto, Calif.). Gene specific primers (5′RACE Primer: 5′ggatggaagccgtgcacatccayacg 3′ (SEQ ID NO: 111 and 3′RACE Primer: 5′ ggcagggaactgacggagagcagg 3′ (SEQ ID NO: 112) were designed according to Heliothis virescens octopamine receptor sequences obtained as described herein above. cDNA libraries generated from Heliothis virescens larval gut were used as template. RACE products were cloned into the TOPO-A vector (Invitrogen Corporation of Carlsbad, Calif.).

Nucleotide Code: n=inosine, Y=C+T, H=A+T+C, B=G+T+C, S=G+C, R=A+G

### Example 4

### Double-Stranded RNA Interference

Preparation of dsRNA for Injection. Sequences to be expressed as dsRNA were cloned into Bluescript KS(+) (Stratagene of La Jolla, Calif.), linearized with the appropriate restriction enzymes, and transcribed in vitro with the Ambion T3 and T7 Megascript kits following the manufacturer's instructions (Ambion Inc. of Austin, Tex.). Transcripts were annealed in injection buffer (0.1 mM NaPO₄ pH 7.8, 5 mM KCl) after heating to 85° C. and cooling to room temperature over a 1- to 24-hr period. All annealed transcripts were analyzed on agarose gels with DNA markers to confirm the size of the annealed RNA and quantitated as described previously (Fire et al. (1998) Nature 391(6669):806-811). Injected RNA was not gel-purified. Injection of 0.1 nl of a 0.1- to 1.0-mg/ml solution of a 1-kb dsRNA corresponds to roughly 10⁷ molecules/injection.

Injection of Drosophila melanogaster Embryos. Fly cages were set up using 2- to 4-day flies. Agar-grape juice plates were replaced every hour to synchronize the egg collection for 1-2 days. The eggs were collected over a 30- to 60-min period for subsequent injection. The eggs were washed into a nylon mesh basket with tap water. The chorion was removed by brief soaking in a dilute bleach solution. Eggs were positioned on a glass slide such that each egg was in a same orientation. Double-stranded RNA was injected into middle of each egg using an Eppendorf transjector (Eppendorf Scientific, Inc. of Westbury, N.Y.). Following injection, slides were stored in a moist chamber to prevent dessication of the embryos. Embryos were monitored for development and transferred as first instar larvae to vials containing Drosophila medium. Methods for rearing Drosophila staging and common genetic techniques can be found, for example, in Roberts (1986) Drosophila melanogaster, A Practical Approach, IRL Press, Washington, D.C.; Ashburner (1989a) Drosophila: A Laboratory Handbook, Cold Spring Harbor Laboratory Press, New York, N.Y.; Ashburner (1989b) Drosophila: A Laboratory Manual, Cold Spring Harbor Laboratory Press, New York, N.Y.; Goldstein & Fyrberg, eds (1994) in Methods in Cell Biology, Vol. 44, Academic Press, San Diego, Calif.

### Example 5

### Expression Pattern Analysis of Drosophila GPCRs by in Situ Hybridization

Wild-type Drosophila melanogaster (strain Oregon R) were obtained from the public Stock Center of Bloomington, Indiana (http://flystocks.bio.indiana.edu). Embryos were collected on grape juice/agar plates at room temperature for approximately 16 hours. Larvae of the wandering 3^{rd} instar stage were dissected such that all internal tissues of the animal were subject to detection. Embryos and larvae were fixed, processed and detected according to the method described by Berleley Drosophila Genome Project website (http://www.fruitfly.org/about/methods/RNAinsitu.html. Following staining, tissues were dissected apart and mounted in 70% Glycerol and examined on a compound microscope. A sense strand control for the wingless gene showed no staining, while the antisense strand displayed the correct expression pattern for wingless. The pattern of expression for each GPCR was recorded by digital photography and are described below in Table 6. Most GPCRs examined have expression in either embryos, larvae or both. Table 6 (GPCR expression) details which GPCRs have expression in which tissues.

TABLE 6
Expression Profile of GPCRs

| | | | larval | larval | | larval | expression |
|---|---|---|---|---|---|---|---|
| | ubiquitous | larval | gut | body wall | larval | Malpighian | in |
| gene | larval | brain | neurons | neurons | gut | tubules | embryos |
| 5-HT1A 56A | x | | | | | | |
| 5-HT2 82C4 | | | | | | | x |
| 5-HT7 100A2 | | | | | x | | |
| Adenosine 99E1 | | | x | x | | | |
| Adrenergic 64C | | x | | | | | |
| Adrenergic 60D1 | | x | | | | | |
| Allatostatin 3E1 | | | | | | | |
| Calcitonin 49F9 | | | | | x | | |
| CCK-X 26A1 | | | | | x | | |
| DiureticHor2 51A1 | | x | | | | | |
| DopR 88B1 | | x | | | | | |
| Galanin 98E2 | | | x | | | x | |
| Gastrin 17E3 | | x | | | x | | |
| GRHR 27A2 | | | | | x | | |
| Histamine 97B | | x | | | x | | |
| He6Receptor 100B1 | | | | | x | | |
| Latrophilin 44D4 | x | | | | | | x |
| Lymnokinin 64D3 | | x | | | | x | |
| mAcR 60C | | x | | | | | |
| Mthlike 1 | | | | | | x | |
| Mthlike 3 | | | | | x | | |
| Neuro Y-like 77A1 | | x | | | | | |
| NeuroYY 26B1 | | | | | x | | |
| Neuro YY-like 83 | | x | x | | x | x | |
| NeuropepYR 97E1 | | x | | | | | |
| Octopamine 90C2 | | | | | x | | |
| Prostaglandin 74F1 | | | | | x | | |
| TakR 86C | | x | | | x | | |
| TakR 99D | | | | | | x | |
| Wormlike 47E | | x | | | x | | |
| Wormlike 4F9 | | x | | | | | |

### Example 6

### Recombinant Production of in E. coli

A cDNA clone of the present invention is subcloned into an appropriate expression vector and transformed into E. coli using the manufacturer's conditions. Specific examples include plasmids such as pBluescript (Stratagene of La Jolla, Calif.), pFLAG (International Biotechnologies, Inc. of New Haven, Connetticut), and pTrcHis (Invitrogen Corp. of Carlsbad, Calif.). E. coli are cultured, expression of the recombinant protein is confirmed, and recombinant protein is isolated using standard techniques.

### Example 7

### Recombinant Production of a GPCR in Insect Cells

Baculovirus vectors, which are derived from the genome of AcNPV virus, are designed to provide high levels of expression of cDNA in the Spodoptera frugiperda (SF9) line of insect cells (ATCC CRL# 1711). Recombinant baculovirus expressing the cDNA of the present invention is produced by the following standard methods (Invitrogen MaxBac Manual, Invitrogen Corporation of Carlsbad, Calif.): cDNA constructs are ligated into the polyhedrin gene in any one of a variety of baculovirus transfer vectors, including the pAC360 and the BleBAc vector (Invitrogen Corp. of Carlsbad, Calif.). Recombinant baculoviruses are generated by homologous recombination following co-transfection of the baculovirus transfer vector and linearized AcNPV genomic DNA (Kitts (1990) Nucleic Acid Res 18:5667) into SF9 cells. Recombinant pAC360 viruses are identified by the absence of inclusion bodies in infected cells and recombinant pBlueBac viruses are identified on the basis of β-galactosidase expression (Summers & Smith, Texas Agriculture Exp Station Bulletin No. 1555).

A cDNA encoding an entire open reading frame for the gene is inserted into the BamH I site of pBlueBacII (Invitrogen Corp. of Carlsbad, Calif.). Constructs in the positive orientation, identified by sequence analysis, are used to transfect SF9 cells in the presence of linear AcNPV wild type DNA. The recombinant insect GPCR is present in the plasma membrane of infected cells. The recombinant insect GPCR is extracted from infected cells by hypotonic or detergent lysis.

### Example 8

### In Vitro Binding Assays

Recombinant protein can be obtained, for example, according to the approach described in Example 6 or 7 herein above. The protein is immobilized on chips appropriate for ligand binding assays. The protein immobilized on the chip is exposed to a candidate substance according to methods known in the art. While the sample compound is in contact with the immobilized protein, measurements capable of detecting protein-ligand interactions are conducted. Measurement techniques include, but are not limited to, SEDLI, Biacore, and FCS, as described above. Substances that bind the protein are readily discovered using this approach and are subjected to further characterization.

### Example 9

### Cell-Based Activity Assay

The cell-based assay is used to detect G-protein Coupled receptor activity, and is performed as essentially described in the protocol provided by Euroscreen (Brussels, Belgium). The GPCR of interest is stably transformed into a recombinant cell line expressing the apoaequorin gene at a high level (AequoScreen™). When the GPCR is activated by an agonist, the heterotrimeric G proteins will activate the enzyme phospholipse Cβ (PLCB). PLCβ then cleaves the phospholipid, phosphatidylinositol 4, 5 bisphosphate (PIP2) to generate diacylglycerol (DAG) and inositol triphosphate (IP3). IP3 binds to the IP3 receptor and releases Ca²⁺ from the intracellular stores. Cells are incubated with coelenterazine, that readily crosses the cell membranes and conjugates to apoaequorin to form aequorin. The activity of the aequorin enzyme is that it emits light upon oxidation of coelenterazine but is dependent on the calcium concentration in its environment. The basal activity of aequorin is very low in the absence of stimulation of the GPCR. When the cells are exposed to an agonist of the GPCR, the rise of intracellular calcium concentration activates the aequorin enzyme, giving a flash luminescence signal. The intensity of the light emission is proportional to the increase in intracellular calcium.

The references cited in the specification are incorporated by reference herein in their entirety.

It will be understood that various details of the invention can be changed without departing from the scope of the invention. Furthermore, the foregoing description is for the purpose of illustration only, and not for the purpose of limitation—the invention being defined by the claims appended hereto.

What is claimed is:

1. An isolated polypeptide comprising:
(a) a polypeptide encoded by the nucleotide sequence of any one of odd numbered sequences SEQ ID NOs:1-107;
(b) a polypeptide encoded by a nucleic acid molecule that is substantially identical to any one of odd numbered sequences SEQ ID NOs:1-107;
(c) a polypeptide comprising the amino acid sequence of any one of even numbered sequences SEQ ID NOs:2-108;
(d) a polypeptide that is a biological equivalent of the polypeptide of any one of even numbered sequences SEQ ID NOs:2-108; or
(e) a polypeptide which is immunologically cross-reactive with an antibody that shows specific binding with a polypeptide of any one of even numbered sequences SEQ ID NOs:2-108.

2. An isolated nucleic acid molecule comprising:
(a) a nucleotide sequence of any one of odd numbered sequences SEQ ID NOs:1-107; or
(b) a nucleic acid molecule substantially identical to any one of odd numbered sequences SEQ ID NOs:1-107.

3. A chimeric gene, comprising the nucleic acid molecule of claim 2 operatively linked to a heterologous promoter.

4. A vector comprising the chimeric gene of claim 3.

5. A host cell comprising the chimeric gene of claim 3.

6. The host cell of claim 5, wherein the cell is selected from the group consisting of a bacterial cell, an insect cell, and a plant cell.

7. A method of detecting a nucleic acid molecule that encodes G-protein coupled receptor polypeptide, the method comprising:
(a) procuring a biological sample comprising nucleic acid material;
(b) hybridizing the nucleic acid molecule of claim 2 under stringent hybridization conditions to the biological sample of (a), thereby forming a duplex structure between the nucleic acid of claim 2 and a nucleic acid within the biological sample; and
(c) detecting the duplex structure of (b), whereby a nuclear receptor nucleic acid molecule is detected.

8. An antibody that specifically recognizes a polypeptide of claim 1.

9. A method for producing an antibody that specifically recognizes a G protein-coupled receptor polypeptide, the method comprising:
(a) recombinantly or synthetically producing an insect nuclear receptor polypeptide, or portion thereof, as set forth in any of even numbered sequences SEQ ID NOs:2-108;
(b) formulating the polypeptide of (a) whereby it is an effective immunogen;
(c) administering to an animal the formulation of (b) to generate an immune response in the animal comprising production of antibodies, wherein antibodies are present in the blood serum of the animal; and
(d) collecting the blood serum from the animal of (c), the blood serum comprising antibodies that specifically recognize a nuclear receptor polypeptide.

10. A method for detecting a level of G protein-coupled receptor polypeptide, the method comprising
(a) obtaining a biological sample comprising peptidic material; and
(b) detecting a G protein-coupled receptor polypeptide in the biological sample of (a) by immunochemical reaction with the antibody of claim 8, whereby a level of G protein-coupled receptor polypeptide in a sample is determined.

11. A method for identifying a substance that modulates G protein-coupled receptor function, the method comprising:
(a) isolating a G protein-coupled receptor polypeptide of claim 1;
(b) exposing the isolated G protein-coupled receptor polypeptide to a plurality of candidate substances;
(c) assaying binding of a candidate substance to the isolated nuclear receptor polypeptide; and
(d) selecting a candidate substance that demonstrates specific binding to the isolated G protein-coupled receptor polypeptide.

12. A method for identifying an insecticidal substance that modulates G protein-coupled receptor function, the method comprising:
(a) isolating a G protein-coupled receptor polypeptide of any one of even numbered SEQ ID NOs:2-108, wherein modulation of the insect G protein -coupled receptor polypeptide confers lethality of an insect during a larval stage;
(b) exposing the isolated G protein-coupled receptor polypeptide to a plurality of substances;
(c) assaying binding of a substance to the isolated G protein-coupled receptor polypeptide; and
(d) selecting a substance that demonstrates specific binding to the isolated G protein-coupled receptor polypeptide.

13. A method for preventing or abrogating an insect infestation of a plant, the method comprising:
(a) preparing an insecticidal composition that includes an G protein -coupled receptor modulator identified according to the method of claim 12; and
(b) contacting an effective dose of the insecticidal composition with a plant, whereby an insect infestation of a plant is prevented or abrogated.

14. The method of claim 13, wherein the insecticidal composition comprises a chemical compound, a protein, a peptide, a nucleic acid, or an antibody.

15. A method for preventing or abrogating a nematode infestation of a plant, the method comprising:
(a) preparing an insecticidal composition that includes a G protein -coupled receptor modulator identified according to the method of claim 12; and
(b) contacting an effective dose of the insecticidal composition with a plant, whereby a nematode infestation of a plant is prevented or abrogated.

16. A method for preventing or abrogating an insect infestation of a plant, the method comprising expressing in a plant a G protein-coupled receptor modulator that modulates the activity of a G protein-coupled receptor polypeptide of claim 1, whereby an insect infestation of the plant is prevented or abrogated.

17. The method of claim 16, wherein the bioactive agent comprises a protein, a peptide, a nucleic acid, or an antibody.

18. A method for preventing or abrogating a nematode infestation of a plant, the method comprising expressing in a plant a bioactive agent that modulates the activity of a G protein-coupled receptor polypeptide of claim 1, whereby a nematode infestation of the plant is prevented or abrogated.

19. A chimeric G protein-coupled receptor cassette comprising a DNA binding domain, a ligand binding domain, and an activation or repression domain, wherein one or more of the DNA binding domain, the ligand binding domain, and the activation domain comprises an amino acid sequence that is identical or substantially identical to a portion of any one of even numbered sequences SEQ ID NOs:2-108.

20. A method of inducing expression of a target nucleotide sequence, the method comprising:
(a) constructing a chimeric G protein-coupled receptor expression cassette of claim 19; and
(b) constructing a target expression cassette having a target nucleotide sequences and a cis-regulatory element that is recognized by a DNA-binding domain of the chimeric G protein-coupled receptor expression cassette;
(c) expressing the chimeric G protein-coupled receptor expression cassette and the target expression cassette in a heterologous organism; and
(d) contacting a ligand that binds to the ligand binding domain of the chimeric G protein-coupled receptor expression cassette with the organism, whereby the target nucleotide sequence is expressed.

21. The method of claim 20, wherein the heterologous organism is a plant.
